(19) United States
(12) Patent Application Publication
(10) Pub. No.: US 2022/0125925 A1
(43) Pub. Date: Apr. 28, 2022

(54) MEDIUM CHAIN TRIGLYCERIDE FORMULATIONS WITH IMPROVED BIOAVAILABILITY AND METHODS RELATED THERETO

(71) Applicant: Cerecin Inc., Denver, CO (US)

(72) Inventors: Samuel T. Henderson, Golden, CO (US); Taryn Boivin, Surry (CA); Judith Walker, Edinburgh (GB)

(73) Assignee: Cerecin Inc., Denver, CO (US)

(21) Appl. No.: 17/436,010

(22) PCT Filed: Mar. 4, 2020
(86) PCT No.: PCT/US20/20976

§ 371 Date: Sep. 2, 2021

Related U.S. Application Data
(60) Provisional application No. 62/813,448, filed on Mar. 4, 2019.
(60) Provisional application No. 62/837,136, filed on Apr. 22, 2019.

(51) Int. Cl. A61K 47/14; A61K 31/19

(52) U.S. Cl. CPC A61K 47/14; A61K 31/19

(57) ABSTRACT

The invention relates compositions of medium chain triglycerides (MCTs), and to methods for treatment with such compositions to treat conditions associated with reduced neuronal metabolism, for example Alzheimer's disease.

### CROSS REFERENCE TO RELATED APPLICATIONS

This application claims the benefit of U.S. Provisional Application No. 62/813,448, filed Mar. 4, 2019, and U.S. Provisional Application No. 62/837,136, filed Apr. 22, 2019, each of the disclosures of which is hereby incorporated by reference in its entirety.

### FIELD OF THE INVENTION

This disclosure relates to pharmaceutical composition comprising high drug loadings of medium chain triglycerides with or without the presence of protein, as well as methods of making and methods of using such compositions.

### BACKGROUND OF THE INVENTION

Medium Chain Triglycerides (MCTs) are comprised of fatty acids with chain length between 5-12 carbons. MCTs have been researched extensively and have known nutritional and pharmaceutical uses. MCTs have melting points which are liquid at room temperature. Further, MCTs are relatively small and are ionizable under physiological conditions, and are generally soluble in aqueous solutions.

When intend to be used as a pharmaceutical composition, it is often desirable to achieve specific pharmacokinetic properties (e.g., Cₘₐₓ, Tₘₐₓ, etc.) based on the intended treatment.

As such, there is a need in the art for pharmaceutical compositions of MCTs that achieve specific pharmacokinetic properties.

### SUMMARY OF THE INVENTION

In an aspect, the disclosure relates to a method of treating a disease or disorder in a subject in need thereof, comprising administering a therapeutically effective amount medium chain triglycerides (MCT), wherein the therapeutically effective amount of MCT is administered in a pharmaceutical composition, wherein the MCT composition provides a maximum concentration (Cₘₐₓ) of total ketones within at least 3 hours after administration, when administered 30 minutes after a standard meal and in the substantial absence of proteins. In some embodiments, the Cₘₐₓ is the maximum serum concentration of total ketones. In some embodiments, the MCT composition provides a maximum serum concentration (Cmax) of total ketones within at least 2.5 hours after administration, at least 2 hours after administration, at least 1.5 hours after administration, or at least 1 hour after administration. In certain embodiments, the therapeutically effective amount of MCT is 20 g and wherein the Cmax of total ketones is at least 400 μmol/L, at least 450 μmol/L, or at least 500 μmol/L. In some embodiments, the MCT pharmaceutical composition is stable at a pH of about 1 to about 3.

In an aspect, the disclosure relates to a method of treating a disease or disorder in a subject in need thereof, comprising administering a therapeutically effective amount medium chain triglycerides (MCT), wherein the therapeutically effective amount of MCT is administered in a pharmaceutical composition, wherein the MCT composition provides a maximum concentration (Cmax) of b-hydroxybutyrate (BHB) within at least 3 hours after administration, when administered 30 minutes after a standard meal and in the substantial absence of proteins. In some embodiments, the Cₘₐₓ is the maximum serum concentration of BHB. In some embodiments, the MCT composition provides a maximum serum concentration (Cmax) of BHB within at least 2.5 hours after administration, at least 2 hours after administration, at least 1.5 hours after administration, or at least 1 hour after administration. In certain embodiments, the therapeutically effective amount of MCT is 20 g and wherein the Cmax of BHB is at least 400 μmol/L, at least 450 μmol/L, or at least 500 μmol/L. In some embodiments, the MCT pharmaceutical composition is stable at a pH of about 1 to about 3.

In an aspect, the disclosure relates to a method of treating a disease or disorder in a subject in need thereof, comprising administering a therapeutically effective amount medium chain triglycerides (MCT), wherein the therapeutically effective amount of MCT is administered in a pharmaceutical composition, wherein the MCT composition provides a maximum concentration (Cₘₐₓ) of acetoacetate (AcAc) within at least 2.5 hours after administration, when administered 30 minutes after a standard meal and in the substantial absence of proteins. In some embodiments, the Cₘₐₓ is the maximum serum concentration of AcAc. In some embodiments, the MCT composition provides a maximum serum concentration (Cmax) of AcAc within at least 2 hours after administration, at least 1.5 hours after administration, or at least 1 hour after administration. In certain embodiments, the therapeutically effective amount of MCT is 20 g and wherein the Cₘₐₓ of AcAc is at least 50 umol/L, at least 60 umol/L, at least 70 umol/L, at least 80 umol/L, at least 90 umol/L, or at least 100 umol/L. In some embodiments, the MCT pharmaceutical composition is stable at a pH of about 1 to about 3.

In an aspect, the disclosure relates to a method of treating a disease or disorder in a subject in need thereof, comprising administering a therapeutically effective amount medium chain triglycerides (MCT), wherein the therapeutically effective amount of MCT is administered in a pharmaceutical composition, wherein the MCT composition provides a maximum concentration (Cₘₐₓ) of total ketones after at least 2.5 hours after administration, when administered 30 minutes after a standard meal and in the presence of proteins. In some embodiments, the Cₘₐₓ is the maximum serum concentration of total ketones. In some embodiments, the MCT composition provides a maximum serum concentration (Cₘₐₓ) of total ketones after at least 3.0 hours after administration, after at least 3.5 hours after administration, after at least 4.0 hours after administration, or after at least 5 hours after administration. In certain embodiments, the therapeutically effective amount of MCT is 20 g and wherein the Cₘₐₓ of total ketones is at least 200 μmol/L, at least 250 μmol/L, at least 300 μmol/L, or at least 350 μmol/L. In some embodiments, the MCT pharmaceutical composition is stable at a pH of about 5 to about 7.

In an aspect, the disclosure relates to a method of treating a disease or disorder in a subject in need thereof, comprising administering a therapeutically effective amount medium chain triglycerides (MCT), wherein the therapeutically effective amount of MCT is administered in a pharmaceutical composition, wherein the MCT composition provides a maximum concentration (Cₘₐₓ) of b-hydroxybutyrate (BHB) after at least 2.5 hours after administration, when administered 30 minutes after a standard meal and in the presence of proteins. In some embodiments, the Cₘₐₓ is the maximum serum concentration of BHB. In some embodiments, the MCT composition provides a maximum serum concentration (Cₘₐₓ) of BHB after at least 3.0 hours after administration, after at least 3.5 hours after administration, after at least 4.0 hours after administration, or after at least 5 hours after administration. In certain embodiments, the therapeutically effective amount of MCT is 20 g and wherein the Cₘₐₓ of BHB is at least 200 μmol/L, at least 250 μmol/L, at least 300 μmol/L, or at least 350 μmol/L. In some embodiments, the MCT pharmaceutical composition is stable at a pH of about 5 to about 7.

In an aspect, the disclosure provides a method of treating a subject in need thereof, comprising administering a therapeutically effective amount medium chain triglycerides (MCT), wherein the therapeutically effective amount of MCT is administered in a pharmaceutical composition, wherein the MCT composition provides a maximum concentration (Cₘₐₓ) of acetoacetate (AcAc) after at least 2.5 hours after administration, when administered 30 minutes after a standard meal and in the presence of proteins. In some embodiments, the Cₘₐₓ is the maximum serum concentration of AcAc. In some embodiments, the MCT composition provides a maximum serum concentration (Cₘₐₓ) of AcAc after at least 3.0 hours after administration, after at least 3.5 hours after administration, after at least 4.0 hours after administration, or after at least 5 hours after administration. In certain embodiments, the therapeutically effective amount of MCT is 20 g and wherein the Cₘₐₓ of AcAc is at least 20 umol/L, at least 25 umol/L, at least 30 umol/L, at least 35 umol/L, or at least 40 umol/L. In some embodiments, the MCT pharmaceutical composition is stable at a pH of about 5 to about 7.

In an aspect, the disclosure provides a method of treating a subject in need thereof, comprising administering a therapeutically effective amount medium chain triglycerides (MCT), wherein the therapeutically effective amount of MCT is administered in two portions, wherein the first portion comprises a first therapeutically effective amount of medium chain triglycerides (MCTs) that is substantially released within 3 hours upon administration to the subject, and wherein the second portion comprises a second therapeutically effective amount of medium chain triglycerides (MCTs) and protein, wherein the second amount of MCTs are substantially released from the second portion over 3 or more hours after administration of the second portion to the subject. In some embodiments, the first portion of MCT is administered in the substantial absence of proteins. In some embodiments, the first portion of MCT is substantially released within 2.5 hours, within 2 hours, within 1.5 hours, or within 1 hour upon administration to the subject. In some embodiments, the second portion of MCT is substantially released over 3.5 or more hours, 4 or more hours, 4.5 or more hours, or 5 or more hours upon administration to the subject.

In some embodiments of the disclosed methods, the disease or disorder is a disease or disorder associated with reduced cognitive function. In certain embodiments of the disclosed methods, the disease or disorder associated with reduced cognitive function is selected from Alzheimer's Disease and Age-Associated memory impairment. In some embodiments of the disclosed methods, the subject lacks the ApoE4 genotype. In certain embodiments of the disclosed methods, the subject is a human.

In some embodiments of the disclose methods, the amount of total ketones, BHB, and/or AcAc is determined using enzymatic methods.

In another aspect, the disclosure provides a pharmaceutical composition comprising a first component and a second component, wherein the first component comprises a therapeutically effective amount of a first portion of medium chain triglycerides (MCTs) that is substantially released within 3 hours upon administration of the pharmaceutical composition to a subject in need thereof, and wherein the second component comprises a therapeutically effective amount of a second portion of medium chain triglycerides (MCTs) and protein, wherein the second portion of MCTs are substantially released from the second component over 3 or more hours after administration of the pharmaceutical composition to the subject. In some embodiments, the first portion of MCTs is substantially released within 2.5 hours, within 2 hours, within 1.5 hours, or within 0.5 hours of administration of the pharmaceutical composition.

In an aspect, the disclosure provides a pharmaceutical composition, comprising: medium chain triglycerides (MCTs) and at least one pharmaceutically acceptable excipient, wherein the composition is substantially free of proteins, wherein the composition provides a maximum concentration (Cₘₐₓ) of at least one ketone body within at least 3 hours after administration, when administered 30 minutes after a standard meal and in the substantial absence of proteins to a subject in need thereof. In some embodiments, the Cₘₐₓ is the maximum serum concentration of the at least one ketone body. In some embodiments, the MCT composition provides a maximum serum concentration (Cₘₐₓ) of at least one ketone body within at least 2.5 hours after administration, at least 2 hours after administration, at least 1.5 hours after administration, or at least 1 hour after administration. The at least one ketone body may be b-hydroxybutyrate (BHB), acetoacetate (AcAc), or combinations thereof.

In some embodiments, the disclosed MCT pharmaceutical composition is an emulsion. In some embodiments, the emulsion does not phase separate for at least 10 minutes, at least 20 minutes, at least 30 minutes, at least 45 minutes, at least 1 hour, at least 2 hours, at least 4 hours, at least 5 hours, or at least 24 hours. In some embodiments, the emulsion has a mean droplet diameter of between about 100 nm and about 1000 nm, about 100 nm and about 500 nm, or about 200 nm and about 300 nm.

In some embodiments, the disclosed MCT pharmaceutical composition of comprises at least 95% tri:C8 MCT. In certain embodiments, the MCT pharmaceutical composition comprises at least 98% tri:C8 MCT.

In some embodiments, the disclosed MCT pharmaceutical composition comprises at least 95% caprylic triglyceride. In some embodiments, the MCT pharmaceutical composition comprises at least 98% caprylic triglyceride.

While multiple embodiments are disclosed, still other embodiments of the present disclosure will become apparent to those skilled in the art from the following detailed description, which shows and describes illustrative embodiments of the disclosure. As will be realized, the invention is capable of modifications in various aspects, all without departing from the spirit and scope of the present disclosure. Accordingly, the detailed descriptions are to be regarded as illustrative in nature and not restrictive.

### BRIEF DESCRIPTION OF THE FIGURES

FIG. 1 shows a graph measuring the serum levels of β-hydroxybutyrate (BHB) in the blood samples versus time after orally administering one of the four formulation containing MCT.

FIG. 2 shows a graph measuring the difference in pharmacokinetic (PK) levels of the serum total ketone levels in the blood samples versus time (hours) after orally administering MCT with water, MCT with Ensure, and MCT with protein.

FIG. 3A shows a graph measuring the baseline-adjusted total ketone levels in serum versus time for each cohort after administering a 20 g dose of MCT with and without food.

FIG. 3B shows a graph measuring observed total ketone levels in serum versus time for each cohort after administering a 20 g dose of MCT with and without food.

FIG. 3C shows a graph measuring the baseline-adjusted β-hydroxybutyrate (BHB) levels versus time in serum for each cohort after administering a 20 g dose of MCT with and without food.

FIG. 3D shows a graph measuring the observed β-hydroxybutyrate (BHB) levels in serum versus time for each cohort after administering a 20 g dose of MCT with and without food.

FIG. 3E shows a graph measuring the baseline-adjusted acetoacetate (AcAc) levels in serum versus time for each cohort after administering a 20 g dose of MCT with and without food.

FIG. 3F shows a graph measuring the observed acetoacetate (AcAc) levels in serum versus time for each cohort after administering a 20 g dose of MCT with and without food.

FIG. 4A shows a graph measuring the observed mean serum total ketone concentrations from an enzymatic assay versus time in the protein based MCT formulations versus non-protein based MCT formulations.

FIG. 4B shows a graph measuring the observed mean serum total ketone baseline-adjusted concentrations from an enzymatic assay versus time in the protein based MCT formulations versus non-protein based MCT formulations.

FIG. 5 shows a table indicating the observed total ketone concentrations from an enzymatic assay in the protein based MCT formulations versus non-protein based MCT formulations.

FIG. 6A shows a graph measuring the observed mean serum β-hydroxybutyrate (BHB) concentrations from an enzymatic assay versus time in the protein based MCT formulations versus non-protein based MCT formulations.

FIG. 6B shows a graph measuring the observed mean serum β-hydroxybutyrate (BHB) base-line adjusted concentrations versus time in the protein based MCT formulations versus non-protein based MCT formulations.

FIG. 7A shows a graph observed mean serum acetoacetate (AcAc) concentrations versus time in the protein based MCT formulations versus non-protein based MCT formulations.

FIG. 7B shows a graph measuring the observed mean serum acetoacetate (AcAc) baseline-adjusted concentrations versus time in the protein based MCT formulations versus non-protein based MCT formulations.

### DETAILED DESCRIPTION OF THE INVENTION

Described herein are several definitions. Such definitions are meant to encompass grammatical equivalents. Unless otherwise required by context, singular terms as used herein and in the claims shall include pluralities and plural terms shall include the singular. The use of “or” means “and/or” unless stated otherwise. Furthermore, the use of the terms “comprising,” “having,” “including,” as well as other forms, such as “includes” and “included,” are intended to be inclusive and mean that there may be additional elements other than the listed elements. Also, terms such as “element” or “component” encompass both elements and components comprising one unit and elements and components that comprise more than one subunit unless specifically stated otherwise.

By way of background, Medium Chain Triglycerides (“MCT”s) are metabolized differently from the more common Long Chain Triglycerides (LCTs). In particular, when compared to LCTs, MCTs are more readily digested to release medium chain fatty acids (MCFAs), which exhibit increased rates of portal absorption, and undergo obligate oxidation. The small size and decreased hydrophobicity of MCTs increases the rate of digestion and absorption relative to LCTs. When MCTs are ingested, they are first processed by lipases, which cleave the fatty acid chains from the glycerol backbone. Some lipases in the pre-duodenum preferentially hydrolyze MCTs over LCTs, and the released MCFAs are then partly absorbed directly by the stomach mucosa. Those MCFAs which are not absorbed in the stomach are absorbed directly into the portal vein and not packaged into lipoproteins. Since blood transports much more rapidly than lymph, MCFAs quickly arrive at the liver. In the liver MCFAs undergo obligate oxidation.

In contrast, long chain fatty acids (LCFAs) derived from normal dietary fat are re-esterified into LCTs and packaged into chylomicrons for transport in the lymph. This greatly slows the metabolism of LCTs relative to MCTs. In the fed state LCFAs undergo little oxidation in the liver, due mainly to the inhibitory effects of malonyl-CoA. When conditions favor fat storage, malonyl-CoA is produced as an intermediate in lipogenesis. Malonyl-CoA allosterically inhibits carnitine palmitoyltransferase I, and thereby inhibits LCFA transport into the mitochondria. This feedback mechanism prevents futile cycles of lipolysis and lipogenesis.

MCFAs are, to a large extent, immune to the regulations that control the oxidation of LCFAs. MCFAs enter the mitochondria without the use of carnitine palmitoyltransferase I, therefore MCFAs by-pass this regulatory step and are oxidized regardless of the metabolic state of the organism. Importantly, since MCFAs enter the liver rapidly and are quickly oxidized, large amounts of ketone bodies are readily produced from MCFAs. As such, a large oral dose of MCTs (e.g., about 20 mL to 40 mL) will result in sustained hyperketonemia.

In certain aspects of the disclosure it has been unexpectedly found that bioavailability of MCTs can be controlled by administering the MCTs with or without proteins. Without intending to be limited, as illustrated herein, it has been found that MCT formulations comprising or being administered with protein provides for a slow or delayed release of the MCT, as compared to administration with low/no protein. Protein-based formulations tend to delay and reduce the maximum (or peak) concentration (“Cₘₐₓ”). Whereas, administration of MCT formulations that are substantially protein-free or administration of MCT the absence of protein allows for maximizing Cₘₐₓ and minimizing the time to reach Cₘₐₓ (“Tₘₐₓ”). In other embodiments, it was found that the bioavailability of MCTs and in vivo formation of active metabolite ketone bodies may be optimized through selective formulation with and without protein carrier excipients.

In certain aspects, it was unexpectedly found that improved bioavailability of MCTs and in vivo formation of active metabolite ketone bodies may be achieved with MCT formulations having a low or no protein content. In this regard, the MCT formulations of the disclosure may be prepared in the substantial absence of proteins. Further, the MCT formulation of the disclosure may be administered in the substantial absence of proteins.

As used herein, “administration” includes an in vivo use environment, such as the gastrointestinal tract, delivery by ingestion or swallowing or other such means to deliver the pharmaceutical composition, as understood by those skilled in the art. See for example, Remington: The Science and Practice of Pharmacy, 20th Edition (2000). Where the aqueous use environment is in vitro, “administration” refers to placement or delivery of the pharmaceutical composition in the in vitro test medium.

As used herein, the terms “substantially protein-free,” “substantially free of protein,” “no protein,” “absence of protein,” “substantial absence of protein,” and the like, refer to the absence of protein in an amount that would meaningfully interfere with the release of MCT. As will be appreciated by one of skill in the art, trace amounts of protein (such as minor contamination) may be present during administration of the MCT or in the MCT formulation without affecting the overall release and metabolism of the MCT and without departing from the spirit of the disclosure with respect to formulations and administration under protein-free conditions. Further, when the MCT formulation is described as administered in the substantial absence of proteins, it is meant that the MCT formulation itself is substantially protein-free and that at the time of administration, substantially no other proteins are administered concurrently with the MCT formulation.

In some embodiments, an MCT formulation that comprises and/or is administered with substantially no protein may provide faster release of the MCT with a higher Cₘₐₓ as compared to an MCT formulation that comprises and/or is administered with protein. In some embodiments, administration an MCT formulation that comprises and/or is administered with substantially no protein may provide an immediate release (IR) of the MCT.

Certain embodiments of the disclosure relate to administering MCT formulations in the substantial absence of protein-based drinks (e.g., Ensure and similar protein-based drink and nutrition supplements). In further embodiments, MCT formulations may be administered in the substantial absence of protein containing foods.

In certain embodiments, substantially no proteins are administered or consumed by the subject about 30 minutes prior to the administration of the MCT formulation. In other embodiments, substantially no proteins are administered or consumed by the subject at least 30 minutes prior to the administration of the MCT formulation. In other embodiments, substantially no proteins are administered or consumed by the subject about 1 hour prior to the administration of the MCT formulation. In other embodiments, substantially no proteins are administered or consumed by the subject at least 1 hour prior to the administration of the MCT formulation.

In some embodiments, substantially no proteins are administered or consumed by the subject during the 30 minutes after administration of the MCT formulation. In other embodiments, substantially no proteins are administered or consumed by the subject at least 30 minutes after administration of the MCT formulation. In other embodiments, substantially no proteins are administered or consumed by the subject during the 1 hour after of administration of the MCT formulation. In other embodiments, substantially no proteins are administered or consumed by the subject at least 1 hour after administration of the MCT formulation. In other embodiments, substantially no proteins are administered or consumed by the subject during the 90 minutes after administration of the MCT formulation. In other embodiments, substantially no proteins are administered or consumed by the subject at least 90 minutes after administration of the MCT formulation. In other embodiments, substantially no proteins are administered or consumed by the subject during the 2 hours after administration of the MCT formulation. In other embodiments, substantially no proteins are administered or consumed by the subject at least 2 hours after administration of the MCT formulation.

In certain embodiments, substantially no proteins are administered or consumed by the subject about 30 minutes prior to the administration of the MCT formulation and about 30 minutes after the administration of the MCT formulation. In other embodiments, substantially no proteins are administered or consumed by the subject at least 30 minutes prior to the administration of the MCT formulation and at least 30 minutes after the administration of the MCT formulation. In other embodiments, substantially no proteins are administered or consumed by the subject about 30 minutes prior to the administration of the MCT formulation and about 1 hour after the administration of the MCT formulation. In other embodiments, substantially no proteins are administered or consumed by the subject at least 30 minutes prior to the administration of the MCT formulation and at least 1 hour after the administration of the MCT formulation. In other embodiments, substantially no proteins are administered or consumed by the subject about 1 hour prior to the administration of the MCT formulation and about 1 hour after the administration of the MCT formulation. In other embodiments, substantially no proteins are administered or consumed by the subject at least 1 hour prior to the administration of the MCT formulation and at least 1 hour after the administration of the MCT formulation.

In one embodiment, the disclosed MCT formulation that comprises and/or is administered with substantially no protein results in elevating ketone concentrations in the body. The MCT formulation may be administered in an amount that is effective to induce hyperketonemia. In one embodiment, hyperketonemia results in ketone bodies being utilized for energy in the brain.

In one embodiment, the disclosed MCT formulation that comprises and/or is administered with substantially no protein increases the circulating concentration of at least one type of ketone body in the subject. The amount of circulating ketone bodies can be measured at a number of times post administration, and in one embodiment, is measured at a time predicted to be near the peak concentration (Cₘₐₓ) in the serum and/or plasma, but can also be measured before or after the predicted peak serum and/or plasma concentration level. Measured amounts at these off-peak times are then optionally adjusted to reflect the predicted level at the predicted peak time. In one embodiment, the predicted peak serum and/or plasma concentration of at least one type of ketone body is at about 0.5 to about 3.0 hours. In another embodiment, the predicted peak serum and/or plasma concentration of at least one ketone body is at about 1.0 to about 2 hours. Peak serum and/or plasma concentrations and timing can vary depending on factors known to those of skill in the art, including individual digestive rates, co-ingestion or pre- or post-ingestion of foods, drinks, etc., as known to one of skill in the art. As will be appreciated by those of skill in the art, other methods besides measuring serum and/or plasma levels can be used to determine levels of ketones; for example by measurement of ketone urinary excretion.

In one embodiment, the disclosed MCT formulation that comprises and/or is administered with substantially no protein increases the circulating concentration of the total ketone bodies in the subject. The disclosed MCT formulation that comprises and/or is administered with substantially no protein may increase the concentration of total ketone bodies as compared to a MCT formulation that comprises and/or is administered with protein.

In an embodiment, the disclosed MCT formulation that comprises and/or is administered with substantially no protein may increase the peak plasma concentration (Cₘₐₓ) of total ketone bodies as compared to a MCT formulation that comprises and/or is administered with protein. In an embodiment, the disclosed MCT formulation that comprises and/or is administered with substantially no protein may increase the peak serum concentration (Cₘₐₓ) of total ketone bodies as compared to a MCT formulation that comprises and/or is administered with protein.

In an embodiment, the peak serum concentration (Cₘₐₓ) of total ketone bodies reached by the disclosed MCT formulation that comprises and/or is administered with substantially no protein is about 10% greater, about 15% greater, about 20% greater, about 25% greater, about 30% greater, about 35% greater, about 40% greater, about 45% greater, about 50% greater, about 55% greater, about 60% greater, about 65% greater, about 70% greater, about 75% greater, about 80% greater, about 85% greater, about 90% greater, about 95% greater, or about 100% greater than the Cₘₐₓ of a MCT formulation that comprises and/or is administered with protein. In some embodiments, the peak serum concentration (Cₘₐₓ) of total ketone bodies reached by the disclosed MCT formulation that comprises and/or is administered with substantially no protein is at least 10% greater, at least 15% greater, at least 20% greater, at least 25% greater, at least 30% greater, at least 35% greater, at least 40% greater, at least 45% greater, at least 50% greater, at least 55% greater, at least 60% greater, at least 65% greater, at least 70% greater, at least 75% greater, at least 80% greater, at least 85% greater, at least 90% greater, at least 95% greater, or at least 100% greater than the Cₘₐₓ of a MCT formulation that comprises and/or is administered with protein.

In an embodiment, the peak serum concentration (Cₘₐₓ) reached of total ketones for the disclosed MCT formulation that comprises and/or is administered with substantially no proteins is between about 350 micromole/liter (μmol/L) to about 1000 μmol/L. In other embodiments, the peak serum concentration (Cₘₐₓ) of total ketone bodies is from about 350 to about 950 μmol/L, from about 350 to about 900 μmol/L, from about 350 to about 850 μmol/L, from about 350 to about 800 μmol/L, from about 350 to about 750 μmol/L, from about 350 to about 700 μmol/L, from about 350 to about 650 μmol/L, from about 350 to about 550 μmol/L, from about 350 to about 500 μmol/L, or from about 350 to about 800 μmol/L, although variations will necessarily occur depending on the composition and subject, for example, as discussed above. In other embodiments, the peak serum concentration (Cₘₐₓ) of total ketone bodies is from about 400 to about 950 μmol/L, from about 400 to about 900 μmol/L, from about 400 to about 850 μmol/L, from about 400 to about 800 μmol/L, from about 400 to about 750 μmol/L, from about 400 to about 700 μmol/L, from about 400 to about 650 μmol/L, from about 400 to about 600 μmol/L, or from about 400 to about 550 μmol/L. In some embodiments, the peak serum concentration (Cₘₐₓ) of total ketone bodies is about 400 to about 600 μmol/L. In other embodiments, the peak serum concentration (Cₘₐₓ) of total ketone bodies is about 450 to about 550 μmol/L. In other embodiments, the peak serum concentration (Cₘₐₓ) of total ketone bodies is at least 350 μmol/L, at least 400 μmol/L, at least 450 μmol/L, at least 500 μmol/L at least 550 μmol/L, or at least 600 μmol/L.

In an embodiment, the disclosed MCT formulation that comprises and/or is administered with substantially no protein results in a time to reach Cₘₐₓ (Tₘₐₓ) of total ketone bodies (Tₘₐₓ) that is less than a MCT formulation comprising and/or administered with protein.

In an embodiment, the time to reach Cₘₐₓ (Tₘₐₓ) of total ketone bodies of the disclosed MCT formulation that comprises and/or is administered with substantially no protein is about 15 minutes, about 30 minutes, about 45 minutes, about 1 hour, about 1.5 hours, about 2 hours, about 2.5 hours, or about 3 hours less than a MCT formulation comprising and/or administered with protein. In some embodiments, the time to reach Cₘₐₓ(Tₘₐₓ) of total ketone bodies of the disclosed MCT formulation that comprises and/or is administered with substantially no protein is at least 15 minutes, at least 30 minutes, at least 45 minutes, at least 1 hour, at least 1.5 hours, at least 2 hours, at least 2.5 hours, or at least 3 hours less than a MCT formulation comprising and/or administered with protein. In some embodiments, the time to reach Cₘₐₓ (Tₘₐₓ) of total ketone bodies of the disclosed MCT formulation that comprises and/or is administered with substantially no protein is at least 30 minutes less than a MCT formulation comprising and/or administered with protein.

In an embodiment, the time to reach Cₘₐₓ (Tₘₐₓ) of total ketone bodies for the disclosed MCT formulation that comprises and/or is administered with substantially no proteins is about 0.5 hour to about 3 hours. In another embodiment, the time to reach Cₘₐₓ(Tₘₐₓ) of total ketone bodies is about 1 hour to about 2.5 hours. In another embodiment, the time to reach Cₘₐₓ (Tₘₐₓ) of total ketone bodies is about 1 hour to about 2 hours. In another embodiment, the time to reach Cₘₐₓ (Tₘₐₓ) is about 0.5 hour to about 1.5 hours. In another embodiment, the time to reach Cₘₐₓ (Tₘₐₓ) of total ketone bodies is about 0.5 hour, about 1 hour, about 1.5 hours, about 2 hours, about 2.5 hours, or about 3 hours. In another embodiment, the time to reach Cₘₐₓ (Tₘₐₓ) of total ketone bodies is less than 3 hours, less than 2.5 hours, less than 2 hours, less than 1.5 hours, or less than 1 hour. In some embodiments, the time to reach Cₘₐₓ (Tₘₐₓ) of total ketone bodies is about 1 hour. In some embodiments, the time to reach Cₘₐₓ (Tₘₐₓ) of total ketone bodies is about 1.5 hours. In some embodiments, the time to reach Cₘₐₓ (Tₘₐₓ) of total ketone bodies is about 2 hours.

In certain embodiments, the disclosed MCT formulation that comprises and/or is administered with substantially no protein increases the circulating concentration of at least one ketone body. In certain embodiments, the disclosed MCT formulation that comprises and/or is administered with substantially no protein increases the circulating concentration of the ketone body beta-hydroxybutyrate (BHB). In certain embodiments, the disclosed MCT formulation that comprises and/or is administered with substantially no protein increases the circulating concentration of the ketone body acetoacetate (AcAc). The disclosed MCT formulation that comprises and/or is administered with substantially no protein may increase the concentration of at least one ketone body as compared to a MCT formulation that comprises and/or is administered with protein.

In an embodiment, the disclosed MCT formulation that comprises and/or is administered with substantially no protein may increase the peak plasma concentration (Cₘₐₓ) of beta-hydroxybutyrate (BHB) as compared to a MCT formulation that comprises and/or is administered with protein. In an embodiment, the disclosed MCT formulation that comprises and/or is administered with substantially no protein may increase the peak serum concentration (Cₘₐₓ) of beta-hydroxybutyrate (BHB) as compared to a MCT formulation that comprises and/or is administered with protein.

In an embodiment, the peak serum concentration (Cₘₐₓ) of beta-hydroxybutyrate (BHB) reached by the disclosed MCT formulation that comprises and/or is administered with substantially no protein is about 10% greater, about 15% greater, about 20% greater, about 25% greater, about 30% greater, about 35% greater, about 40% greater, about 45% greater, about 50% greater, about 55% greater, about 60% greater, about 65% greater, about 70% greater, about 75% greater, about 80% greater, about 85% greater, about 90% greater, about 95% greater, or about 100% greater than the Cₘₐₓ of a MCT formulation that comprises and/or is administered with protein. In some embodiments, the peak serum concentration (Cₘₐₓ) of beta-hydroxybutyrate (BHB) reached by the disclosed MCT formulation that comprises and/or is administered with substantially no protein is at least 10% greater, at least 15% greater, at least 20% greater, at least 25% greater, at least 30% greater, at least 35% greater, at least 40% greater, at least 45% greater, at least 50% greater, at least 55% greater, at least 60% greater, at least 65% greater, at least 70% greater, at least 75% greater, at least 80% greater, at least 85% greater, at least 90% greater, at least 95% greater, or at least 100% greater than the Cₘₐₓ of a MCT formulation that comprises and/or is administered with protein.

In one embodiment, the peak serum concentration (Cₘₐₓ) reached of beta-hydroxybutyrate (BHB) for the disclosed MCT formulation that comprises and/or is administered with substantially no proteins is between about 350 micromole/liter (μmol/L) to about 1000 μmol/L. In other embodiments, the peak serum concentration (Cₘₐₓ) of beta-hydroxybutyrate (BHB) is from about 350 to about 950 μmol/L, from about 350 to about 900 μmol/L, from about 350 to about 850 μmol/L, from about 350 to about 800 μmol/L, from about 350 to about 750 μmol/L, from about 350 to about 700 μmol/L, from about 350 to about 650 μmol/L, from about 350 to about 550 μmol/L, from about 350 to about 500 μmol/L, or from about 350 to about 800 μmol/L, although variations will necessarily occur depending on the composition and subject, for example, as discussed above. In other embodiments, the peak serum concentration (Cₘₐₓ) of beta-hydroxybutyrate (BHB) is from about 400 to about 950 μmol/L, from about 400 to about 900 μmol/L, from about 400 to about 850 μmol/L, from about 400 to about 800 μmol/L, from about 400 to about 750 μmol/L, from about 400 to about 700 μmol/L, from about 400 to about 650 μmol/L, from about 400 to about 600 μmol/L, or from about 400 to about 550 μmol/L. In some embodiments, the peak serum concentration (Cₘₐₓ) of beta-hydroxybutyrate (BHB) is about 350 to about 600 μmol/L. In other embodiments, the peak serum concentration (Cₘₐₓ) of beta-hydroxybutyrate (BHB) is about 350 to about 550 μmol/L. In some embodiments, the peak serum concentration (Cₘₐₓ) of beta-hydroxybutyrate (BHB) is about 400 to about 500 μmol/L. In other embodiments, the peak serum concentration (Cₘₐₓ) of beta-hydroxybutyrate (BHB) is at least 350 μmol/L, at least 400 μmol/L, at least 450 μmol/L, at least 500 μmol/L at least 550 μmol/L, or at least 600 μmol/L.

In one embodiment, the disclosed MCT formulation that comprises and/or is administered with substantially no protein results in a time to reach Cₘₐₓ (Tₘₐₓ) of beta-hydroxybutyrate (BHB) that is less than the MCT formulation comprising and/or administered with protein.

In an embodiment, the time to reach Cₘₐₓ (Tₘₐₓ) of beta-hydroxybutyrate (BHB) of the disclosed MCT formulation that comprises and/or is administered with substantially no protein is about 15 minutes, about 30 minutes, about 45 minutes, about 1 hour, about 1.5 hours, about 2 hours, about 2.5 hours, or about 3 hours less than a MCT formulation comprising and/or administered with protein. In some embodiments, the time to reach Cₘₐₓ(Tₘₐₓ) of beta-hydroxybutyrate (BHB) of the disclosed MCT formulation that comprises and/or is administered with substantially no protein is at least 15 minutes, at least 30 minutes, at least 45 minutes, at least 1 hour, at least 1.5 hours, at least 2 hours, at least 2.5 hours, or at least 3 hours less than a MCT formulation comprising and/or administered with protein. In some embodiments, the time to reach Cₘₐₓ (Tₘₐₓ) of beta-hydroxybutyrate (BHB) of the disclosed MCT formulation that comprises and/or is administered with substantially no protein is at least 30 minutes than a MCT formulation comprising and/or administered with protein.

In one embodiment the time to reach Cₘₐₓ (Tₘₐₓ) of beta-hydroxybutyrate (BHB) for the disclosed MCT formulation that comprises and/or is administered with substantially no proteins is about 0.5 hour to about 3 hours. In another embodiment, the time to reach Cₘₐₓ(Tₘₐₓ) of beta-hydroxybutyrate (BHB) is about 1 hour to about 2.5 hours. In another embodiment, the time to reach Cₘₐₓ (Tₘₐₓ) of beta-hydroxybutyrate (BHB) is about 1 hour to about 2 hours. In another embodiment, the time to reach Cₘₐₓ (Tₘₐₓ) of beta-hydroxybutyrate (BHB) is about 0.5 hour to about 1.5 hours. In another embodiment, the time to reach Cₘₐₓ(Tₘₐₓ) of beta-hydroxybutyrate (BHB) is about 0.5 hour, about 1 hour, about 1.5 hours, about 2 hours, about 2.5 hours, or about 3 hours. In another embodiment, the time to reach Cₘₐₓ(Tₘₐₓ) of beta-hydroxybutyrate (BHB) is less than 3 hours, less than 2.5 hours, less than 2 hours, less than 1.5 hours, or less than 1 hour. In some embodiments, the time to reach Cₘₐₓ(Tₘₐₓ) of beta-hydroxybutyrate (BHB) is about 1 hour. In some embodiments, the time to reach Cₘₐₓ (Tₘₐₓ) of beta-hydroxybutyrate (BHB) is about 1.5 hours. In some embodiments, the time to reach Cₘₐₓ (Tₘₐₓ) of beta-hydroxybutyrate (BHB) is about 2 hours.

In an embodiment, the disclosed MCT formulation that comprises and/or is administered with substantially no protein may increase the peak plasma concentration (Cₘₐₓ) of acetoacetate (AcAc) as compared to a MCT formulation that comprises and/or is administered with protein. In an embodiment, the disclosed MCT formulation that comprises and/or is administered with substantially no protein may increase the peak serum concentration (Cₘₐₓ) of acetoacetate (AcAc) as compared to a MCT formulation that comprises and/or is administered with protein.

In an embodiment, the peak serum concentration (Cₘₐₓ) of acetoacetate (AcAc) reached by the disclosed MCT formulation that comprises and/or is administered with substantially no protein is about 10% greater, about 15% greater, about 20% greater, about 25% greater, about 30% greater, about 35% greater, about 40% greater, about 45% greater, about 50% greater, about 55% greater, about 60% greater, about 65% greater, about 70% greater, about 75% greater, about 80% greater, about 85% greater, about 90% greater, about 95% greater, or about 100% greater than the Cₘₐₓ of a MCT formulation that comprises and/or is administered with protein. In some embodiments, the peak serum concentration (Cₘₐₓ) of acetoacetate (AcAc) reached by the disclosed MCT formulation that comprises and/or is administered with substantially no protein is at least 10% greater, at least 15% greater, at least 20% greater, at least 25% greater, at least 30% greater, at least 35% greater, at least 40% greater, at least 45% greater, at least 50% greater, at least 55% greater, at least 60% greater, at least 65% greater, at least 70% greater, at least 75% greater, at least 80% greater, at least 85% greater, at least 90% greater, at least 95% greater, or at least 100% greater than the Cₘₐₓ of a MCT formulation that comprises and/or is administered with protein.

In one embodiment, the peak serum concentration (Cₘₐₓ) reached of acetoacetate (AcAc) for the disclosed MCT formulation that comprises and/or is administered with substantially no proteins is between about 20 micromole/liter (μmol/L) to about 200 μmol/L. In other embodiments, the peak serum concentration (Cₘₐₓ) of acetoacetate (AcAc) is from about 20 to about 180 μmol/L, about 20 to about 160 μmol/L, about 20 to about 140 μmol/L, about 20 to about 120 μmol/L, about 20 to about 100 μmol/L, about 20 to about 80 μmol/L, about 20 to about 60 μmol/L, or about 20 to about 40 μmol/L, although variations will necessarily occur depending on the composition and subject, for example, as discussed above. In other embodiments, the peak serum concentration (Cₘₐₓ) of acetoacetate (AcAc) is from about 40 to about 140 μmol/L, about 40 to about 100 μmol/L, or about 40 to about 80 μmol/L. In other embodiments, the peak serum concentration (Cₘₐₓ) of acetoacetate (AcAc) is from about 60 to about 120 μmol/L. In other embodiments, the peak serum concentration (Cₘₐₓ) of acetoacetate (AcAc) is at least 20 μmol/L, at least 30 μmol/L, at least 40 μmol/L, at least 50 μmol/L, at least 60 μmol/L, at least 70 μmol/L, at least 80 μmol/L, at least 90 μmol/L, at least 90 μmol/L, or at least 100 μmol/L. In other embodiments, the peak serum concentration (Cₘₐₓ) of acetoacetate (AcAc) is at least 80 μmol/L.

In one embodiment, the disclosed MCT formulation that comprises and/or is administered with substantially no protein results in a time to reach Cₘₐₓ (Tₘₐₓ) of beta-acetoacetate (AcAc) that is less than the MCT formulation comprising and/or administered with protein.

In an embodiment, the time to reach Cₘₐₓ (Tₘₐₓ) of acetoacetate (AcAc) of the disclosed MCT formulation that comprises and/or is administered with substantially no protein is about 15 minutes, about 30 minutes, about 45 minutes, about 1 hour, about 1.5 hours, about 2 hours, about 2.5 hours, or about 3 hours less than a MCT formulation comprising and/or administered with protein. In some embodiments, the time to reach Cₘₐₓ(Tₘₐₓ) of acetoacetate (AcAc) of the disclosed MCT formulation that comprises and/or is administered with substantially no protein is at least 15 minutes, at least 30 minutes, at least 45 minutes, at least 1 hour, at least 1.5 hours, at least 2 hours, at least 2.5 hours, or at least 3 hours less than a MCT formulation comprising and/or administered with protein. In some embodiments, the time to reach Cₘₐₓ (Tₘₐₓ) of acetoacetate (AcAc) of the disclosed MCT formulation that comprises and/or is administered with substantially no protein is at least 30 minutes than a MCT formulation comprising and/or administered with protein.

In one embodiment the time to reach Cₘₐₓ (Tₘₐₓ) acetoacetate (AcAc) is about 0.5 hour to about 3 hours for the disclosed MCT formulation that comprises and/or is administered with substantially no proteins. In another embodiment, the time to reach Cₘₐₓ(Tₘₐₓ) of acetoacetate (AcAc) is about 1 hour to about 2.5 hours. In another embodiment, the time to reach Cₘₐₓ (Tₘₐₓ) of acetoacetate (AcAc) is about 1 hour to about 2 hours. In another embodiment, the time to reach Cₘₐₓ (Tₘₐₓ) of acetoacetate (AcAc) is about 0.5 hour to about 1.5 hours. In another embodiment, the time to reach Cₘₐₓ (Tₘₐₓ) of acetoacetate (AcAc) is about 0.5 hour, about 1 hour, about 1.5 hours, about 2 hours, about 2.5 hours, or about 3 hours. In another embodiment, the time to reach Cₘₐₓ (Tₘₐₓ) of acetoacetate (AcAc) is less than 3 hours, less than 2.5 hours, less than 2 hours, less than 1.5 hours, or less than 1 hour. In some embodiments, the time to reach Cₘₐₓ (Tₘₐₓ) of acetoacetate (AcAc) is about 1 hour. In some embodiments, the time to reach Cₘₐₓ (Tₘₐₓ) of acetoacetate (AcAc) is about 1.5 hours. In some embodiments, the time to reach Cₘₐₓ (Tₘₐₓ) of acetoacetate (AcAc) is about 2 hours.

In other embodiments of the disclosure, an MCT formulation that comprises and/or is administered with protein may provide slower release of the MCT. In some embodiments, administration an MCT formulation that comprises and/or is administered with low/no protein may provide a sustained release (SR), delayed release (DR), and/or controlled (CR) of the MCT.

As will be appreciated by those of skill in the art, the amount of protein administered with the MCT formulation and/or present in the MCT formulation may be varied to achieve a desired release profile of the MCT. For example, lower amounts of protein may provide a faster sustained/delayed/controlled release as compared to higher amounts of protein.

In certain embodiments, proteins are administered or consumed by the subject concurrently with the administration of the MCT formulation. In other embodiments, proteins are administered or consumed by the subject at the same time as the administration of the MCT formulation. In certain embodiments, proteins are administered or consumed by the subject less than 30 minutes prior to the administration of the MCT formulation. In other embodiments, proteins are administered or consumed by the subject less than 30 minutes after the administration of the MCT formulation. In certain embodiments, proteins are administered or consumed by the subject less than 30 minutes prior to the administration of the MCT formulation and less than 30 minutes after the administration of the MCT formulation. In certain embodiments, proteins are administered or consumed by the subject 15 minutes or less prior to the administration of the MCT formulation. In other embodiments, proteins are administered or consumed by the subject 15 minutes or less after the administration of the MCT formulation. In certain embodiments, proteins are administered or consumed by the subject 15 minutes or less prior to the administration of the MCT formulation and 15 minutes or less after the administration of the MCT formulation.

In one embodiment, the disclosed MCT formulation that comprises and/or is administered with protein results in elevating ketone concentrations in the body, which are less than the ketone concentrations of a MCT formulation comprising and/or administered with substantially no protein. The MCT formulation may be administered in an amount that is effective to induce hyperketonemia. In one embodiment, hyperketonemia results in ketone bodies being utilized for energy in the brain.

The disclosed MCT formulation that comprises and/or is administered with protein may have lower the concentration of total ketone bodies as compared to a MCT formulation that comprises and/or is administered with substantially no protein.

In an embodiment, the disclosed MCT formulation that comprises and/or is administered with protein may decrease the peak plasma concentration (Cₘₐₓ) of total ketone bodies as compared to a MCT formulation that comprises and/or is administered with substantially no protein. In an embodiment, the disclosed MCT formulation that comprises and/or is administered with protein may decrease the peak serum concentration (Cₘₐₓ) of total ketone bodies as compared to a MCT formulation that comprises and/or is administered with substantially no protein.

In an embodiment, the peak serum concentration (Cₘₐₓ) of total ketone bodies reached by the disclosed MCT formulation that comprises and/or is administered with protein is about 10% lower, about 15% lower, about 20% lower, about 25% lower, about 30% lower, about 35% lower, about 40% lower, about 45% lower, about 50% lower, about 55% lower, about 60% lower, about 65% lower, about 70% lower, about 75% lower, about 80% lower, about 85% lower, about 90% lower, about 95% lower, or about 100% lower than the Cₘₐₓ of a MCT formulation that comprises and/or is administered with substantially no protein. In some embodiments, the peak serum concentration (Cₘₐₓ) of total ketone bodies reached by the disclosed MCT formulation that comprises and/or is administered with protein is at least 10% lower, at least 15% lower, at least 20% lower, at least 25% lower, at least 30% lower, at least 35% lower, at least 40% lower, at least 45% lower, at least 50% lower, at least 55% lower, at least 60% lower, at least 65% lower, at least 70% lower, at least 75% lower, at least 80% lower, at least 85% lower, at least 90% lower, at least 95% lower, or at least 100% lower than the Cₘₐₓ of a MCT formulation that comprises and/or is administered with substantially no protein.

In one embodiment, the disclosed MCT formulation that comprises and/or is administered with protein results in a time to reach Cₘₐₓ (Tₘₐₓ) of total ketones that is greater than the MCT formulation comprising and/or administered with substantially no protein.

In an embodiment, the time to reach Cₘₐₓ (Tₘₐₓ) of total ketone bodies of the disclosed MCT formulation that comprises and/or is administered with protein is about 15 minutes, about 30 minutes, about 45 minutes, about 1 hour, about 1.5 hours, about 2 hours, about 2.5 hours, or about 3 hours greater than a MCT formulation comprising and/or administered with substantially no protein. In some embodiments, the time to reach Cₘₐₓ(Tₘₐₓ) of total ketone bodies of the disclosed MCT formulation that comprises and/or is administered with protein is at least 15 minutes, at least 30 minutes, at least 45 minutes, at least 1 hour, at least 1.5 hours, at least 2 hours, at least 2.5 hours, or at least 3 hours greater than a MCT formulation comprising and/or administered with substantially no protein. In some embodiments, the time to reach Cₘₐₓ (Tₘₐₓ) of total ketone bodies of the disclosed MCT formulation that comprises and/or is administered with protein is at least 60 minutes greater than greater than a MCT formulation comprising and/or administered with substantially no protein.

In certain embodiments, the disclosed MCT formulation that comprises and/or is administered with protein increases the circulating concentration of at least one ketone body. In certain embodiments, the disclosed MCT formulation that comprises and/or is administered with protein increases the circulating concentration of the ketone body beta-hydroxybutyrate (BHB). In certain embodiments, the disclosed MCT formulation that comprises and/or is administered with protein increases the circulating concentration of the ketone body acetoacetate (AcAc). The disclosed MCT formulation that comprises and/or is administered with protein may decrease the concentration of at least one ketone body as compared to a MCT formulation that comprises and/or is administered with substantially no protein.

In an embodiment, the disclosed MCT formulation that comprises and/or is administered with protein may decrease the peak plasma concentration (Cₘₐₓ) of beta-hydroxybutyrate (BHB) as compared to a MCT formulation that comprises and/or is administered with substantially no protein. In an embodiment, the disclosed MCT formulation that comprises and/or is administered with protein may decrease the peak serum concentration (Cₘₐₓ) of beta-hydroxybutyrate (BHB) as compared to a MCT formulation that comprises and/or is administered with substantially no protein.

In an embodiment, the peak serum concentration (Cₘₐₓ) of beta-hydroxybutyrate (BHB) reached by the disclosed MCT formulation that comprises and/or is administered with protein is about 10% lower, about 15% lower, about 20% lower, about 25% lower, about 30% lower, about 35% lower, about 40% lower, about 45% lower, about 50% lower, about 55% lower, about 60% lower, about 65% lower, about 70% lower, about 75% lower, about 80% lower, about 85% lower, about 90% lower, about 95% lower, or about 100% lower than the Cₘₐₓ of a MCT formulation that comprises and/or is administered with substantially no protein. In some embodiments, the peak serum concentration (Cₘₐₓ) of beta-hydroxybutyrate (BHB) reached by the disclosed MCT formulation that comprises and/or is administered with protein is at least 10% lower, at least 15% lower, at least 20% lower, at least 25% lower, at least 30% lower, at least 35% lower, at least 40% lower, at least 45% lower, at least 50% lower, at least 55% lower, at least 60% lower, at least 65% lower, at least 70% lower, at least 75% lower, at least 80% lower, at least 85% lower, at least 90% lower, at least 95% lower, or at least 100% lower than the Cₘₐₓ of a MCT formulation that comprises and/or is administered with substantially no protein.

In one embodiment, the disclosed MCT formulation that comprises and/or is administered with protein results in a time to reach Cₘₐₓ (Tₘₐₓ) of beta-hydroxybutyrate (BHB) that is greater than the MCT formulation comprising and/or administered with substantially no protein.

In an embodiment, the time to reach Cₘₐₓ (Tₘₐₓ) of beta-hydroxybutyrate (BHB) of the disclosed MCT formulation that comprises and/or is administered with protein is about 15 minutes, about 30 minutes, about 45 minutes, about 1 hour, about 1.5 hours, about 2 hours, about 2.5 hours, or about 3 hours greater than a MCT formulation comprising and/or administered with substantially no protein. In some embodiments, the time to reach Cₘₐₓ(Tₘₐₓ) of total ketone bodies of the disclosed MCT formulation that comprises and/or is administered with protein is at least 15 minutes, at least 30 minutes, at least 45 minutes, at least 1 hour, at least 1.5 hours, at least 2 hours, at least 2.5 hours, or at least 3 hours greater than a MCT formulation comprising and/or administered with substantially no protein. In some embodiments, the time to reach Cₘₐₓ (Tₘₐₓ) of total ketone bodies of the disclosed MCT formulation that comprises and/or is administered with protein is at least 60 minutes greater than greater than a MCT formulation comprising and/or administered with substantially no protein.

In an embodiment, the disclosed MCT formulation that comprises and/or is administered with protein may decrease the peak plasma concentration (Cₘₐₓ) of acetoacetate (AcAc) as compared to a MCT formulation that comprises and/or is administered with substantially no protein. In an embodiment, the disclosed MCT formulation that comprises and/or is administered with protein may decrease the peak serum concentration (Cₘₐₓ) of acetoacetate (AcAc) as compared to a MCT formulation that comprises and/or is administered with substantially no protein.

In an embodiment, the peak serum concentration (Cₘₐₓ) of acetoacetate (AcAc) reached by the disclosed MCT formulation that comprises and/or is administered with protein is about 10% lower, about 15% lower, about 20% lower, about 25% lower, about 30% lower, about 35% lower, about 40% lower, about 45% lower, about 50% lower, about 55% lower, about 60% lower, about 65% lower, about 70% lower, about 75% lower, about 80% lower, about 85% lower, about 90% lower, about 95% lower, or about 100% lower than the Cₘₐₓ of a MCT formulation that comprises and/or is administered with substantially no protein. In some embodiments, the peak serum concentration (Cₘₐₓ) of acetoacetate (AcAc) reached by the disclosed MCT formulation that comprises and/or is administered with protein is at least 10% lower, at least 15% lower, at least 20% lower, at least 25% lower, at least 30% lower, at least 35% lower, at least 40% lower, at least 45% lower, at least 50% lower, at least 55% lower, at least 60% lower, at least 65% lower, at least 70% lower, at least 75% lower, at least 80% lower, at least 85% lower, at least 90% lower, at least 95% lower, or at least 100% lower than the Cₘₐₓ of a MCT formulation that comprises and/or is administered with substantially no protein.

In one embodiment, the disclosed MCT formulation that comprises and/or is administered with protein results in a time to reach Cₘₐₓ (Tₘₐₓ) of beta-acetoacetate (AcAc) that is greater than the MCT formulation comprising and/or administered with substantially no protein.

In an embodiment, the time to reach Cₘₐₓ (Tₘₐₓ) of acetoacetate (AcAc) of the disclosed MCT formulation that comprises and/or is administered with protein is about 15 minutes, about 30 minutes, about 45 minutes, about 1 hour, about 1.5 hours, about 2 hours, about 2.5 hours, or about 3 hours greater than a MCT formulation comprising and/or administered with substantially no protein. In some embodiments, the time to reach Cₘₐₓ(Tₘₐₓ) of total ketone bodies of the disclosed MCT formulation that comprises and/or is administered with protein is at least 15 minutes, at least 30 minutes, at least 45 minutes, at least 1 hour, at least 1.5 hours, at least 2 hours, at least 2.5 hours, or at least 3 hours greater than a MCT formulation comprising and/or administered with substantially no protein. In some embodiments, the time to reach Cₘₐₓ (Tₘₐₓ) of total ketone bodies of the disclosed MCT formulation that comprises and/or is administered with protein is at least 60 minutes greater than greater than a MCT formulation comprising and/or administered with substantially no protein.

It will be appreciated by those of skill in the art, that analysis of the ketone bodies measurements/quantification can be, in some circumstances, adjusted to account for error, baseline measurements, etc. The amount of one or more ketone bodies may be determined from whole blood, plasma, serum, and or combinations thereof. The amount of one or more ketone bodies maybe determine by methods known to those of skill, including, but not limited to enzymatic assays and liquid chromatography-tandem mass spectrometry (LC-MS).

In yet other embodiments of the disclosure, an MCT formulation that comprises a combination of components, one with low/no protein and one with protein may provide: a combination IR and SR/DR/CR pharmacokinetic profile, with the protein based component providing an IR phase, and the low/no protein component providing a SR/DR/CR phase.

In some embodiments, the MCT formulation may comprise at least two components—the first component allowing for an IR pharmacokinetic profile and a second component allowing for a SR/DR/CR pharmacokinetic profile. Each of the components may comprise a therapeutically effective amount of MCT. The amount of MCT in each component is readily ascertainable by one of skill in the art based on the desired results and pharmacokinetic profile and the disease/disorder to be treated, and the characteristics of the intended subject or subject population.

In an embodiment, the first component (IR) may be substantially free of protein and the second component (SR/DR/CR) may contain protein. To maintain the IR pharmacokinetic profile, the MCT formulation may be administered with substantially no protein. In some embodiments, substantially no proteins are administered or consumed by the subject about 30 minutes prior to the administration of the MCT formulation. In other embodiments, substantially no proteins are administered or consumed by the subject at least 30 minutes prior to the administration of the MCT formulation. In other embodiments, substantially no proteins are administered or consumed by the subject about 1 hour prior to the administration of the MCT formulation. In other embodiments, substantially no proteins are administered or consumed by the subject at least 1 hour prior to the administration of the MCT formulation. In some embodiments, substantially no proteins are administered or consumed by the subject during the 30 minutes after administration of the MCT formulation. In other embodiments, substantially no proteins are administered or consumed by the subject at least 30 minutes after administration of the MCT formulation. In other embodiments, substantially no proteins are administered or consumed by the subject during the 1 hour after of administration of the MCT formulation. In other embodiments, substantially no proteins are administered or consumed by the subject at least 1 hour after administration of the MCT formulation. In other embodiments, substantially no proteins are administered or consumed by the subject during the 90 minutes after administration of the MCT formulation. In other embodiments, substantially no proteins are administered or consumed by the subject at least 90 minutes after administration of the MCT formulation.

In an embodiment, the second component (SR/DR/CR) may contain protein. The second component (SR/DR/CR) may be formulated such that it does not interfere with the immediate release of the first component (IR). In some embodiments, the first component (IR) comprises a therapeutically effective amount of a first portion of MCTs that is substantially released within 1 hour, 1.5 hours, 2 hours, 2.5 hours, or 3 hours upon administration of the MCT formulation. In an embodiment, the first component comprises a therapeutically effective amount of a first portion of MCTs that is substantially released within 1 hour upon administration of the MCT formulation. In an embodiment, the first component comprises a therapeutically effective amount of a first portion of MCTs that is substantially released within 2 hours upon administration of the MCT formulation. In an embodiment, the first component comprises a therapeutically effective amount of a first portion of MCTs that is substantially released within 3 hours upon administration of the MCT formulation. In some embodiments, the second component (SR/DR/CR) comprises a therapeutically effective amount of a second portion of MCTs that is substantially released over 2 or more hours, 2.5 or more hours, 3 or more hours, 3.5 or more hours, 4.0 or more hours, 4.5 or more hours, or 5 or more hours upon administration of the MCT formulation. In an embodiment, the first component comprises a therapeutically effective amount of a second portion of MCTs that is substantially released over 2 or more hours. In an embodiment, the second component comprises a therapeutically effective amount of a second portion of MCTs that is substantially released over 3 or more hours. In an embodiment, the second component comprises a therapeutically effective amount of a first portion of MCTs that is substantially released over 4 or more hours.

In in some embodiments, the first component (IR) maybe administered to the subject separately from the second component (SR/DR/CR). As such, another aspect of this disclosure is a method of administering a first component (IR) comprising MCT substantially in the absence of protein and then administering a second component (SR/DR/CR) comprising both MCTs and protein. In another embodiment, disclosed is a method of administering a first component (IR) comprising MCTs substantially in the absence of protein and then administering a second component (SR/DR/CR) comprising MCTs with protein. In an embodiment, the first and second components may be administered about 30 minutes, about 45 minutes, about 1 hour, about 1.5 hours, about 2 hours, about 2.5 hours, or about 3 hours apart. In an embodiment, the first and second components may be administered at least 30 minutes, at least 45 minutes, at least 1 hour, at least 1.5 hours, at least 2 hours, at least 2.5 hours, or at least 3 hours apart.

In another embodiment, the second component is administered at least minutes after the first component and is administered with protein. The second component may comprise the protein, or protein may be administered with the second component. When the protein is administered with the second component, it may be administered within at least 15 minutes, at least 30 minutes, at least 45 minutes, or at least 60 minutes of the second component.

The present disclosure further generally relates to pharmaceutical compositions comprising a high loading of an active agent comprising at least one MCT, and methods of making and using such compositions. As discussed, in some embodiments the MCT formulation is substantially free of protein. In other embodiments, the MCT formulation may contain protein.

In an embodiment, it was unexpectedly found that that improved bioavailability of MCTs and in vivo formation of active metabolite ketone bodies may be achieved with MCT formulations that form stable emulsions of high drug loadings of MCTs, both in the presence of and in the absence of proteins.

In certain aspects, the MCT formulations of the disclosure form stable emulsions on reconstitution in an aqueous use environment, e.g., in water or when administered in vivo.

In certain embodiments, the emulsion formed does not phase separate for the duration of stability. By way of example, the emulsions may be stable for at least about 10 minutes, at least about 20 minutes, at least about 30 minutes, at least about 45 minutes at least about 1 hour, at least about 2 hours, at least about 4 hours, at least about 5 hours, at least about 24 hours, etc.

In certain aspects, the emulsions may generally be stable at ambient conditions and neutral pH, and at physiological conditions upon administration (e.g., at body temperature and physiological pH conditions in the stomach).

In certain embodiments, the emulsion formed may be stable at stomach pH, e.g., at a pH of about 1 to about 3, about 1.2 to 2.9, etc. In certain embodiments, the emulsion formed may be stable at intestinal and/or colon pH, e.g., at a pH of about 5 to about 7, about 5.5 to about 6.9, etc. In certain embodiments, the emulsion formed may begin to break down or phase separate at stomach pH after about ½ to about 1 hour, but does not release the encapsulated MCT until intestinal or colon pH. In this regard, without intending to be limited by theory, in-vitro digestion assays indicate that encapsulated MCT is released from emulsion at intestinal and/or colon pH, which is the primary location of lipid digestion enzymes. In accordance with certain aspects of the disclosure, preferential release of MCT in the intestines and/or colon rather than the stomach may increase bioavailability of the MCT given the location of lipid digestion enzymes in these areas.

In certain embodiments, the emulsion formed may begin to break down or phase separate at stomach pH after about 0.5 to about 1 hour, but does not release the absorbed/encapsulated or otherwise included high drug loading of MCT until intestinal or colon pH.

In this regard, certain aspects of the disclosure are directed to the preferential release of high drug loaded MCTs in the intestines and/or colon rather than the stomach. Without intending to the be limited by theory, this preferential release in the intestines and/or colon may increase bioavailability of the MCTs given that the colon is a primary location of lipid digestion enzymes as compared to the stomach.

In certain aspects, the MCT formulations generate a stable emulsion having a small mean droplet particle size. The small mean droplet particle size generates large relative surface area of emulsion droplets within the emulsion. Without intending to be limited by theory, this large relative surface area of emulsion droplets provides a large surface area for lipid digestion enzymes to act to release adsorbed/encapsulated or otherwise incorporated MCT and to thereby breakdown the MCT to active metabolite ketone bodies. As such, the larger the relative surface area of emulsion droplets, the larger the amount of MCT available for lipid digestion enzymes action, and thereby the greater the amount of generated active metabolite ketone bodies. In certain embodiments, the emulsions may have a mean droplet diameter of less than about 1000 nm, but greater than about 100 nm, e.g. between about 100 nm and 500 nm, between about 200 nm and about 300 nm, etc.

In other aspects of the disclosure, and again without intending to be limited by theory, preferential release of MCTs in the colon may provide reduced stomach upset and related adverse events as compared to standard administration of non-formulated MCT oil.

In yet other aspects of the disclosure, as described above, the improved bioavailability of the MCTs may generally lead to increased active metabolite ketone body production in vivo, as compared to standard administration of non-formulated MCT oil or as compared to administration of MCT formulated with and/or administered with protein.

In summary, stable emulsions of MCTs comprising small emulsion droplets will provide good bioavailability of the MCT, in part because high surface area emulsion droplets facilitate efficient digestion of MCTs to active metabolite ketone bodies by lipid digestion enzymes in the colon. Without being limited by theory, MCT formulations comprising or administered with protein coagulates at stomach pH, thereby causing the formulation to breakdown the emulsion and release the API into the stomach. Such formulations are generally less available to colon lipid digestion enzymes, and thereby the MCT is less bioavailable

A MCT formulation that comprises and/or is administered with low/no protein and that forms a stable emulsion at ambient and physiological conditions including stomach pH provide: higher bioavailability vs protein based MCT formulations, lower dosage volume, and reduced AE's, therefore, reduced patient titration period.

In certain embodiments, the pharmaceutical compositions may include a high drug load of an active agent comprising or consisting essentially of at least one MCT, such as caprylic triglyceride, of at least about 30% by weight of the total composition, at least about 35% of the total composition, at least about 40% by weight of the total composition, about 30% by weight of the total composition to about 65% by weight of the total composition, about 30% by weight of the total composition to about 60% by weight of the total composition, about 35% by weight of the total composition to about 60% by weight of the total composition about 40% by weight of the total composition to about 55% by weight of the total composition, about 40% by weight of the total composition to about 50% by weight of the total composition, etc.

As used herein, unless otherwise specified, “% by weight” refers to “% by weight of the total composition”.

In certain aspects, the solid pharmaceutical compositions of the disclosure may comprise a high drug loading of an active agent comprising or consisting essentially of at least one MCT, at least one or two surfactants, an adsorbent, and a film forming polymer. The pharmaceutical compositions may also include a co-surfactant.

In certain aspects of the disclosure, MCTs refer to any glycerol molecule ester-linked to three fatty acid molecules, each fatty acid molecule having a carbon chain of 5-12 carbons. In certain embodiments, the pharmaceutical compositions may comprise an MCT represented by the following general formula:

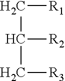

wherein R₁, R₂ and R₃ are fatty acids having 5-12 carbons in the carbon backbone esterified to the glycerol backbone.

The MCTs of the disclosure may be prepared by any process known in the art, such as direct esterification, rearrangement, fractionation, transesterification, or the like. Sources of the MCT include any suitable source, semi-synthetic, synthetic or natural. Examples of natural sources of MCT include plant sources such as coconuts and coconut oil, palm kernels and palm kernel oils, and animal sources such as milk from any of a variety of species, e.g., goats. For example, the lipids may be prepared by the rearrangement of a vegetable oil such as coconut oil. The length and distribution of the chain length may vary depending on the source oil. For example, MCT containing 1-10% C6, 30-60% C8, 30-60% C10, 1-10% C10 are commonly derived from palm and coconut oils.

In accordance with certain embodiments of the disclosure, the solid pharmaceutical compositions of the disclosure may comprise an active agent comprising or consisting essentially of MCTs that have greater than about 95%, e.g., 98%, C8 at R₁, R₂ and R₃, and are herein referred to as caprylic triglyceride (“CT”).

In certain embodiments, the MCT is caprylic triglyceride, as described herein. Exemplary sources of CT include MIGLYOL® 808 or NEOBEE® 895. In certain aspects, CT may be obtained from coconut or palm kernel oil, made by semi-synthetic esterification of octanoic acid to glycerin, etc.

In other embodiments, the solid pharmaceutical compositions may comprise an active agent comprising or consisting essentially of MCTs wherein R₁, R₂, and R₃ are fatty acids containing a six-carbon backbone (tri-C6:0). Tri-C6:0 MCT are absorbed very rapidly by the gastrointestinal tract in a number of animal model systems. The high rate of absorption results in rapid perfusion of the liver, and a potent ketogenic response. In another embodiment, the pharmaceutical compositions may comprise an active agent comprising or consisting essentially of MCTs wherein R₁, R₂, and R₃ are fatty acids containing an eight-carbon backbone (tri-C8:0). In another embodiment, the pharmaceutical compositions may comprise an active agent comprising or consisting essentially of MCTs wherein R₁, R₂, and R₃ are fatty acids containing a ten-carbon backbone (tri-C10:0). In another embodiment, the pharmaceutical compositions may comprise MCTs wherein R₁, R₂, and R₃ are a mixture of C8:0 and C10:0 fatty acids. In another embodiment, the pharmaceutical compositions may comprise an active agent comprising or consisting essentially of MCTs wherein R₁, R₂ and R₃ are a mixture of C6:0, C8:0, C10:0, and C12:0 fatty acids. In another embodiment, the pharmaceutical compositions may comprise an active agent comprising or consisting essentially of MCTs wherein greater than 95% of R₁, R₂ and R₃ are 8 carbons in length. In yet another embodiment, the pharmaceutical compositions may comprise an active agent comprising or consisting essentially of MCTs wherein the R₁, R₂, and R₃ carbon chains are 6-carbon or 10-carbon chains. In another embodiment, the pharmaceutical compositions may comprise an active agent comprising or consisting essentially of MCTs wherein about 50% of R₁, R₂ and R₃ are 8 carbons in length and about 50% of R₁, R₂ and R₃ 10 carbons in length. In one embodiment, the pharmaceutical compositions may comprise an active agent comprising or consisting essentially of MCTs wherein R₁, R₂ and R₃ are 6, 7, 8, 9, 10 or 12 carbon chain length, or mixtures thereof.

As discussed, in some embodiments of the present disclosure relate to MCT formulation comprises at least two MCT containing components—at least one component allowing for an IR pharmacokinetic profile and at least one component allowing for a SR/DR/CR pharmacokinetic profile. As will be appreciated by one of skill, multi component MCT formulations may be determined by those skilled in the art in view of the present disclosure.

In certain aspects, the disclosure relates to methods of treating a disease or disorder associated with reduced cognitive function in a subject in need thereof, the method comprising administering to the subject a pharmaceutical composition of the disclosure in an amount effective to elevate ketone body concentrations in said subject to thereby treat said disease or disorder. In certain embodiments, the pharmaceutical composition of the disclosure may be administered outside of the context of a ketogenic diet. For instance, in the context of the present disclosure, carbohydrates may be consumed at the same time as pharmaceutical compositions disclosed herein.

In accordance with certain aspects of the disclosure, diseases and disorders associated with reduced cognitive function including Age-Associated Memory Impairment (AAMI), Alzheimer's Disease (AD), Parkinson's Disease, Friedreich's Ataxia (FRDA), GLUT1-deficient Epilepsy, Leprechaunism, and Rabson-Mendenhall Syndrome, Coronary Arterial Bypass Graft (CABG) dementia, anesthesia-induced memory loss, Huntington's Disease, and many others.

In another embodiment, the patient has or is at risk of developing disease-related reduced cognitive function caused by reduced neuronal metabolism, for example, reduced cognitive function associated with Alzheimer's Disease (AD), Parkinson's Disease, Friedreich's Ataxia (FRDA), GLUT-1-deficient Epilepsy, Leprechaunism, and Rabson-Mendenhall Syndrome, Coronary Arterial Bypass Graft (CABG) dementia, anesthesia-induced memory loss, Huntington's Disease, and many others.

In another embodiment, the subject, lacks the ApoE4 genotype as described in U.S. Pat. No. 8,445,535, the entirety of which is hereby incorporated by reference.

As used herein, reduced neuronal metabolism refers to all possible mechanisms that could lead to a reduction in neuronal metabolism. Such mechanisms include, but are not limited to mitochondrial dysfunction, free radical attack, generation of reactive oxygen species (ROS), ROS-induced neuronal apoptosis, defective glucose transport or glycolysis, imbalance in membrane ionic potential, dysfunction in calcium flux, and the like.

According to the present invention, high blood ketone levels will provide an energy source for brain cells that have compromised glucose metabolism, leading to improved performance in cognitive function. As used herein, “subject” and “patient” are used interchangeably, and refer to any mammal, including humans that may benefit from treatment of disease and conditions associated with or resulting from reduced neuronal metabolism.

“Effective amount” refers to an amount of a compound, material, or pharmaceutical composition, as described herein that is effective to achieve a particular biological result. Effectiveness for treatment of the aforementioned conditions may be assessed by improved results from at least one neuropsychological test. These neuropsychological tests are known in the art and include Clinical Global Impression of Change (CGIC), Rey Auditory Verbal Learning Test (RAVLT), First-Last Names Association Test (FLN), Telephone Dialing Test (TDT), Memory Assessment Clinics Self-Rating Scale (MAC-S), Symbol Digit Coding (SDC), SDC Delayed Recall Task (DRT), Divided Attention Test (DAT), Visual Sequence Comparison (VSC), DAT Dual Task (DAT Dual), Mini-Mental State Examination (MMSE), and Geriatric Depression Scale (GDS), among others.

The term “cognitive function” refers to the special, normal, or proper physiologic activity of the brain, including, without limitation, at least one of the following: mental stability, memory/recall abilities, problem solving abilities, reasoning abilities, thinking abilities, judging abilities, capacity for learning, perception, intuition, attention, and awareness. “Enhanced cognitive function” or “improved cognitive function” refers to any improvement in the special, normal, or proper physiologic activity of the brain, including, without limitation, at least one of the following: mental stability, memory/recall abilities, problem solving abilities, reasoning abilities, thinking abilities, judging abilities, capacity for learning, perception, intuition, attention, and awareness, as measured by any means suitable in the art. “Reduced cognitive function” or “impaired cognitive function” refers to any decline in the special, normal, or proper physiologic activity of the brain.

In another embodiment, the methods of the present invention further comprise determination of the patients' genotype or particular alleles. In one embodiment, the patient's alleles of the apolipoprotein E gene are determined. It has been found that non-E4 carriers performed better than those with the E4 allele when elevated ketone body levels were induced with MCT. Also, those with the E4 allele had higher fasting ketone body levels and the levels continued to rise at the two hour time interval. Therefore, E4 carriers may require higher ketone levels or agents that increase the ability to use the ketone bodies that are present.

In one embodiment, the pharmaceutical compositions of the disclosure are administered orally. Therapeutically effective amounts of the therapeutic agents can be any amount or dose sufficient to bring about the desired effect and depend, in part, on the severity and stage of the condition, the size and condition of the patient, as well as other factors readily known to those skilled in the art. The dosages can be given as a single dose, or as several doses, for example, divided over the course of several weeks, as discussed elsewhere herein.

The pharmaceutical compositions of the disclosure, in one embodiment, are administered in a dosage required to increase blood ketone bodies to a level required to treat and/or prevent the occurrence of any disease- or age-associated cognitive decline, such as AD, AAMI, and the like. Appropriate dosages may be determined by one of skill in the art.

In one embodiment, oral administration of a pharmaceutical composition of the disclosure results in hyperketonemia. Hyperketonemia, in one embodiment, results in ketone bodies being utilized for energy in the brain even in the presence of glucose. Additionally, hyperketonemia results in a substantial (39%) increase in cerebral blood flow (Hasselbalch, S. G., et al., Changes in cerebral blood flow and carbohydrate metabolism during acute hyperketonemia, Am J Physiol, 1996, 270:E746-51). Hyperketonemia has been reported to reduce cognitive dysfunction associated with systemic hypoglycemia in normal humans (Veneman, T., et al., Effect of hyperketonemia and hyperlacticacidemia on symptoms, cognitive dysfunction, and counterregulatory hormone responses during hypoglycemia in normal humans, Diabetes, 1994, 43:1311-7). Please note that systemic hypoglycemia is distinct from the local defects in glucose metabolism that occur in any disease- or age-associated cognitive decline, such as AD, AAMI, and the like.

Administration can be on an as-needed or as-desired basis, for example, once-monthly, once-weekly, daily, or more than once daily. Similarly, administration can be every other day, week, or month, every third day, week, or month, every fourth day, week, or month, and the like. Administration can be multiple times per day. When utilized as a supplement to ordinary dietetic requirements, the composition may be administered directly to the patient or otherwise contacted with or admixed with daily feed or food.

The pharmaceutical compositions provided herein are, in one embodiment, intended for “long term” consumption, sometimes referred to herein as for ‘extended’ periods. “Long term” administration as used herein generally refers to periods in excess of one month. Periods of longer than two, three, or four months comprise one embodiment of the instant invention. Also included are embodiments comprising more extended periods that include longer than 5, 6, 7, 8, 9, or 10 months. Periods in excess of 11 months or 1 year are also included. Longer terms use extending over 1, 2, 3 or more years are also contemplated herein. “Regular basis” as used herein refers to at least weekly, dosing with or consumption of the compositions. More frequent dosing or consumption, such as twice or thrice weekly are included. Also included are regimens that comprise at least once daily consumption. The skilled artisan will appreciate that the blood level of ketone bodies, or a specific ketone body, achieved may be a valuable measure of dosing frequency. Any frequency, regardless of whether expressly exemplified herein, that allows maintenance of a blood level of the measured compound within acceptable ranges can be considered useful herein. The skilled artisan will appreciate that dosing frequency will be a function of the composition that is being consumed or administered, and some compositions may require more or less frequent administration to maintain a desired blood level of the measured compound (e.g., a ketone body).

Administration can be carried out on a regular basis, for example, as part of a treatment regimen in the patient. A treatment regimen may comprise causing the regular ingestion by the patient of a pharmaceutical composition of the disclosure in an amount effective to enhance cognitive function, memory, and behavior in the patient. Regular ingestion can be once a day, or two, three, four, or more times per day, on a daily or weekly basis. Similarly, regular administration can be every other day or week, every third day or week, every fourth day or week, every fifth day or week, or every sixth day or week, and in such a regimen, administration can be multiple times per day. The goal of regular administration is to provide the patient with optimal dose of a pharmaceutical composition of the disclosure, as exemplified herein.

Dosages of the inventive compositions, such as, for example, those comprising MCT, may be administered in an effective amount to increase the cognitive ability of patients afflicted with diseases of reduced neuronal metabolism, such as in patients with any disease- or age-associated cognitive decline, such as, AD, AAMI, and the like.

Effective amount of dosages of compounds for the inventive compositions, i.e., compounds capable of elevating ketone body concentrations in an amount effective for the treatment of or prevention of loss of cognitive function caused by reduced neuronal metabolism will be apparent to those skilled in the art. As discussed herein above, such effective amounts can be determined in light of disclosed blood ketone levels. Where the compound capable of elevating ketone body concentrations is MCT, the MCT dose, in one embodiment, is in the range of about 0.05 g/kg/day to about 10 g/kg/day of MCT. In other embodiments, the dose will be in the range of about 0.25 g/kg/day to about 5 g/kg/day of MCT. In other embodiments, the dose will be in the range of about 0.5 g/kg/day to about 2 g/kg/day of MCT. In other embodiments, the dose will be in the range of about 0.1 g/kg/day to about 2 g/kg/day. In other embodiments, the dose of MCT is at least about 0.05 g/kg/day, at least about 0.1 g/kg/day, at least about 0.15 g/kg/day, at least about 0.2 g/kg/day, at least about 0.5 g/kg/day, at least about 1 g/kg/day, at least about 1.5 g/kg/day, at least about 2 g/kg/day, at least about 2.5 g/kg/day, at least about 3 g/kg/day, at least about 4 g/kg/day, at least about 5 g/kg/day, at least about 10 g/kg/day, at least about 15 g/kg/day, at least about 20 g/kg/day, at least about 30 g/kg/day, at least about 40 g/kg/day, and at least about 50 g/kg/day.

Convenient unit dosage containers and/or compositions include sachets or containers of spray dried particles, tablets, capsules, lozenges, troches, hard candies, nutritional bars, nutritional drinks, metered sprays, creams, and suppositories, among others. The compositions may be combined with a pharmaceutically acceptable excipient such as gelatin, oil(s), and/or other pharmaceutically active agent(s). Some examples of compositions are described in WIPO Publication 2008/170235, the entirety of which is incorporated by reference. For example, the compositions may be advantageously combined and/or used in combination with other therapeutic or prophylactic agents, different from the subject compounds. In many instances, administration in conjunction with the subject compositions enhances the efficacy of such agents. For example, the compounds may be advantageously used in conjunction with antioxidants, compounds that enhance the efficiency of glucose utilization, and mixtures thereof.

The daily dose of MCT can also be measured in terms of grams of MCT per kg of body weight (BW) of the mammal. The daily dose of MCT can range from about 0.01 g/kg to about 10.0 g/kg BW of the mammal. Preferably, the daily dose of MCT is from about 0.1 g/kg to about 5 g/kg BW of the mammal. More preferably, the daily dose of MCT is from about 0.2 g/kg to about 3 g/kg of the mammal. Still more preferably, the daily dose of MCT is from about 0.5 g/kg to about 2 g/kg of the mammal.

In some embodiments, the inventive compounds may be administered in the substantial absence of protein, or be co-formulated without protein.

In some embodiments, the MCT formulation may be co-administered with protein, or be co-formulated with protein.

In some embodiments, the MCT formulation may be co-administered with protein, or be co-formulated with protein. Protein can include more than one type of protein or protein different from one or more sources. Appropriate proteins are known in the art. If co-formulated, the amount of protein to use can include at least about 0.1 g, at least about 1 g, at least about 10 g, at least about 50 g, at least about 100 g, at least about 150 g, at least about 200 g, at least about 250 g, at least about 300 g, at least about 400 g. Amounts of protein can be at least about 1 g, at least about 50 g, at least about 100 g. The compositions can comprise from about 15% to about 40% protein, on a dry weight basis. Sources of such proteins include legumes, grains, dairy, nuts, seeds, fruits, vegetables, animals, insects, synthetic sources (e.g., genetically modified yeast), or mixtures thereof. The compositions also optionally comprise other components that comprise protein such as dried whey and other dairy products or by-products. In some embodiments the MCT formulations are administered in the presence of protein-based drinks (e.g., Ensure and similar protein-based drink and nutrition supplements).

Additionally, in some embodiments, the MCT formulation may be co-administered with carbohydrate, or be co-formulated with carbohydrate. Carbohydrate can include more than one type of carbohydrate. Appropriate carbohydrates are known in the art, and include simple sugars, such as glucose, fructose, sucrose, and the like, from conventional sources such as corn syrup, sugar beet, and the like. If co-formulated, the amount of carbohydrate to use can include at least about 0.1 g, at least about 1 g, at least about 10 g, at least about 50 g, at least about 100 g, at least about 150 g, at least about 200 g, at least about 250 g, at least about 300 g, at least about 400 g. Amounts of carnitine can be at least about 1 g, at least about 50 g, at least about 100 g. The compositions can comprise from about 15% to about 40% carbohydrate, on a dry weight basis. Sources of such carbohydrates include grains or cereals such as rice, corn, sorghum, alfalfa, barley, soybeans, canola, oats, wheat, or mixtures thereof. The compositions also optionally comprise other components that comprise carbohydrates such as dried whey and other dairy products or by-products.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Comparison of Serum Ketone Levels Resulting from Administration of MCT Formulations, AC-1202, Axona, and AC-1204

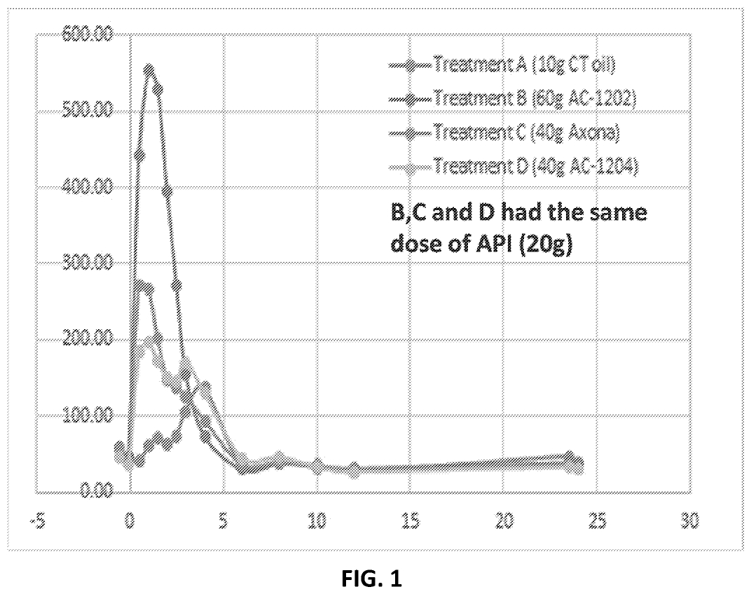

The following study is designed to demonstrate the bioequivalence (BE) of two commercial formulations of medium chain triglycerides and two inventive formulations of medium chain triglycerides (MCT). Subjects (healthy young males) were divided into four separate groups. The following formulations were administered orally to groups: Group 1, treatment A: 10 g of commercially available CT oil; Group 2, treatment B: 60 g AC-1202 (at least 95% tri: C8/gum acacia); Group 3, treatment C (40 g Axona); and Group 4, treatment D AC-1204 (at least 95% tri: C8/gum acacia with protein). Treatments B, C, and D were each dosed at 20 g of the MCT. After administration, blood samples were obtained at various time points from each subject and evaluated using enzymatic methods (Wako Diagnostics) measuring the serum ketone levels (β-hydroxybutyrate, BHB). FIG. 1 shows the comparison of the serum ketone levels in the serum versus time.

As FIG. 1 shows, the two inventive formulations, AC-1202 and AC-1204, achieved higher serum ketone levels (BHB) as compared to the two commercial formulations. AC-1202 achieved a higher level of BHB and a larger Cₘₐₓ as compared to the three other formulations. AC-1204 generated a lower level of BHB and a lower Cₘₐₓ as compared to AC-1202.

### Example 2—Comparison of MCT Formulation AC-1202 Administered with Water or Ensure

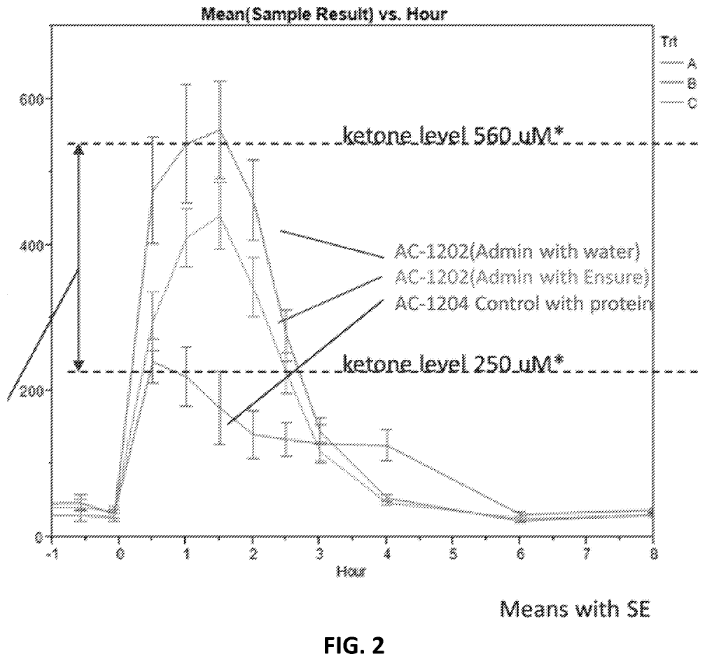

The following study is designed to demonstrate the difference in PK profiles between AC-1202 administered with water, AC-1202 administered with Ensure, and AC-1204 (the control formulation comprising protein). Each subject (healthy young males) were administered one of the formulations orally. After administration, blood samples were obtained at various time points from each subject and evaluated using enzymatic methods (Wako Diagnostics) measuring the serum ketone levels (β-hydroxybutyrate, BHB). FIG. 2 shows the comparison of the serum ketone levels in the serum versus time.

Table 1 depicts the mean BHB in subjects at day 45 and day 90 who were either administered AC-1202 administered with water or AC1202 administered with Ensure. Across all patients, significantly higher ketone body levels were in seen in AC1202+water vs. AC-1202+Ensure.

TABLE 1
Mean BHB Levels

| | Day 45 | | Day 90 | |
|---|---|---|---|---|
| | AC-1202 | AC-1202 | AC-1202 | AC-1202 |
| | with | with | with | with |
| | Ensure | water | Ensure | water |
| Number of | 47 | 16 | 38 | 9 |
| Subjects | | | | |
| Mean BHB | 0.31 | 0.52 | 0.33 | 0.68 |
| Difference | 0.21 | | 0.35 | |
| P value | 0.0025 | | 0.0034 | |

As FIG. 2 and Table 1 demonstrated, AC-1202 formulation administered in water achieved the highest Cₘₐₓ generating a ketone level if 560 μM (mean with SE). The control formulation, AC-1204 generated less than half the serum levels of serum ketone levels verses the AC-1202 administered in water.

### Example 3—Food Effects Study (FES): Administrations of MCT Formulations with High Drug Load with and without Food

The following study is designed to determine a Food Effect Study (FES). MCT formulations administered at a dose of 20 g tricaprilin with and without food, no titration. In this study, two sequential cohorts were examined. Cohort 1 subjects were Caucasians. A 4 way crossover conditions were used with cohort 1. Cohort 2 subjects were Asians. A 2 way crossover conditions were used with cohort 2. The last condition in cohort 2 was dropped based on safety and tolerability based on the results of the cohort 1.

The Table 2 shows the experimental protocol used in this group and the results.

TABLE 2
Experimental Protocol.

| Cohort # | Period | Subjects | Conditions | Results |
|---|---|---|---|---|
| 1 | 1 | Caucasians | MCT with | Not well tolerated |
| | | | Standard Meal | from GI perspective |
| 1 | 2 | Caucasians | MCT 30 minutes | Well tolerated from |
| | | | after standard meal | GI perspective |
| 1 | 3 | Caucasians | MCT 30 minutes | Well tolerated from |
| | | | after high fat meal | GI perspective |
| 1 | 4 | Caucasians | MCT administered | Very poorly tolerated |
| | | | while fasting | from a GI perspective |
| 2 | 1 | Asians | MCT 30 minutes | Well tolerated from |
| | | | after standard meal | GI perspective |
| 2 | 2 | Asians | dropped | NA |

The MCTs were delivered as follows: 50 g of a sprayed dried powder containing the equivalent of 20 g tricaprilin was shaken in approximately 180 mL of water and administered to the subject orally. Immediately after dosing, the remaining treatment in the cup was shaken up with an additional 60 mL of water and administered to the subject for a total of approximately 240 mL of dosing water to be consumed for each dosing. The MCT formulation did not contain protein.

Tables 3-5 show the observed and adjusted pharmacokinetic values (AUC, Cₘₐₓ, Tₘₐₓ) for the cohorts and time periods described in Table 1. Parmacokinetic analysis was performed using noncompartmental analysis and nominal sampling times. Total ketones and BHB serum concentration were determined by enzymatic method. AcAc concentrations were derived by subtracting BHB from total ketones. If the resulting value was negative, it was set to 0. Baseline (BL) adjusted values were determined for total ketones, BHB, and AcAc in Period 1, 2, 3, and 4 using a nominal time-matched correction, where each baseline time point on Period 1 Day −1 was subtracted from the time-matched time point on Day 1. If a nominal time 0 h collection occurred on Day 1, the −1 h Day −1 was used for baseline adjustment. Missing values on either Day −1 or Day 1 resulted in a missing baseline-adjusted value for that time point. Negative values resulting from baseline adjustment were set to 0 for PK parameter calculation and descriptive statistics. Baseline corrected parameters were determined as follows: (1) Cₘₐₓ and Tₘₐₓ were obtained directly from the baseline adjusted concentration-time curve; and (2) partial AUCs were calculated by subtracting the Day −1 Period 1 partial AUC from the Day 1 AUC for each period. Total ketone and BHB concentrations that were BLQ were set to LLOQ/2 for acetoacetate (AcAc) determination, baseline correction, the calculation of descriptive statistics, and PK parameter determination.

Pharmacokinetic parameters were derived using noncompartmental methods with Phoenix® WinNonlin® Version 6.4 or higher (Certata, L. P. Princeton, N.J., United States of America [USA]) and/or SAS® Version 9.2 or higher (SAS Institute, Inc., Cary, N.C., USA).

TABLE 3
Mean and Standard Deviation for Total Ketones PK Parameters.

| | | | | Observed | | | | | BL Adjusted | | | | |
|---|---|---|---|---|---|---|---|---|---|---|---|---|---|
| | | | | AUC0_4 | AUC0_6 | AUC0_8 | Cmax | | AUC0_4 | AUC0_6 | AUC0_8 | Cmax | |
| | | | Sub- | hr*umol/ | hr*umol/ | hr*umol/ | umol/ | Tmax | hr*umol/ | hr*umol/ | hr*umol/ | umol/ | Tmax |
| | | PK_ | ject_ | L | L | L | L | hr | L | L | L | L | hr |
| Cohort | Period | DAY | ID | Estimate | | | | | | | | | |
| 1 | 1 | −1 | Mean | 1470 | 1840 | 2350 | 612 | 4.45 | | | | | |
| | | | SD | 422 | 588 | 749 | 160 | 2.54 | | | | | |
| 1 | 1 | 1 | Mean | 3110 | 3580 | 3850 | 1470 | 0.750 | 1640 | 1740 | 1500 | 1180 | 0.750 |
| | | | SD | 798 | 1130 | 1330 | 266 | 0.264 | 693 | 1060 | 1310 | 193 | 0.264 |
| 1 | 2 | 1 | Mean | 2440 | 2800 | 2980 | 1090 | 1.55 | 968 | 964 | 748 | 718 | 1.25 |
| | | | SD | 513 | 618 | 656 | 185 | 0.762 | 551 | 698 | 696 | 139 | 0.635 |
| 1 | 3 | 1 | Mean | 2920 | 3410 | 3560 | 1190 | 1.85 | 1440 | 1570 | 1230 | 831 | 1.55 |
| | | | SD | 660 | 776 | 803 | 259 | 0.851 | 790 | 880 | 979 | 317 | 0.599 |
| 1 | 4 | 1 | Mean | 2780 | 3350 | 3600 | 1140 | 1.85 | 1310 | 1510 | 1260 | 777 | 1.75 |
| | | | SD | 696 | 848 | 846 | 242 | 0.818 | 490 | 665 | 658 | 171 | 0.791 |
| 2 | 1 | −1 | Mean | 1860 | 2600 | 2950 | 665 | 3.00 | | | | | |
| | | | SD | 1120 | 1470 | 1570 | 328 | 1.13 | | | | | |
| 2 | 1 | 1 | Mean | 3550 | 4210 | 4520 | 1490 | 1.20 | 1690 | 1610 | 1570 | 1120 | 1.05 |
| | | | SD | 1090 | 1560 | 1800 | 252 | 0.675 | 590 | 734 | 835 | 160 | 0.643 |

TABLE 4
Mean and Standard Deviation for BHB PK Parameters.

| | | | | Observed | | | | | BL Adjusted | | | | |
|---|---|---|---|---|---|---|---|---|---|---|---|---|---|
| | | | | AUC0_4 | AUC0_6 | AUC0_8 | Cmax | | AUC0_4 | AUC0_6 | AUC0_8 | Cmax | |
| | | | Sub- | hr*umol/ | hr*umol/ | hr*umol/ | umol/ | Tmax | hr*umol/ | hr*umol/ | hr*umol/ | umol/ | Tmax |
| | | PK_ | ject_ | L | L | L | L | hr | L | L | L | L | hr |
| Cohort | Period | DAY | ID | Estimate | | | | | | | | | |
| 1.00 | 1 | −1 | Mean | 1080 | 1310 | 1670 | 462 | 4.85 | | | | | |
| | | | SD | 355 | 467 | 614 | 143 | 2.77 | | | | | |
| 1.00 | 1 | 1 | Mean | 2170 | 2450 | 2590 | 1100 | 0.750 | 1100 | 1140 | 924 | 891 | 0.750 |
| | | | SD | 589 | 840 | 981 | 217 | 0.264 | 489 | 783 | 966 | 163 | 0.264 |
| 1.00 | 2 | 1 | Mean | 1840 | 2060 | 2150 | 864 | 1.55 | 767 | 770 | 586 | 588 | 1.30 |
| | | | SD | 359 | 436 | 458 | 143 | 0.762 | 437 | 526 | 511 | 94.0 | 0.675 |
| 1.00 | 3 | 1 | Mean | 2130 | 2460 | 2530 | 942 | 1.85 | 1060 | 1150 | 909 | 678 | 1.50 |
| | | | SD | 469 | 547 | 560 | 228 | 0.851 | 598 | 671 | 708 | 268 | 0.577 |
| 1.00 | 4 | 1 | Mean | 2220 | 2630 | 2780 | 951 | 1.85 | 1150 | 1320 | 1110 | 663 | 1.75 |
| | | | SD | 555 | 674 | 671 | 224 | 0.818 | 374 | 529 | 548 | 149 | 0.791 |
| 2.00 | 1 | −1 | Mean | 1330 | 1850 | 2060 | 492 | 3.05 | | | | | |
| | | | SD | 830 | 1110 | 1180 | 257 | 1.23 | | | | | |
| 2.00 | 1 | 1 | Mean | 2520 | 2950 | 3130 | 1100 | 1.20 | 1190 | 1090 | 1070 | 849 | 0.85 |
| | | | SD | 808 | 1120 | 1300 | 221 | 0.753 | 505 | 606 | 670 | 169 | 0.530 |

TABLE 5
Mean and Standard Deviation for Acetoacetate (AcAc) PK Parameters.

| | | | | Observed | | | | | BL Adjusted | | | | |
|---|---|---|---|---|---|---|---|---|---|---|---|---|---|
| | | | | AUC0_4 | AUC0_6 | AUC0_8 | Cmax | | AUC0_4 | AUC0_6 | AUC0_8 | Cmax | |
| | | | Sub- | hr*umol/ | hr*umol/ | hr*umol/ | umol/ | Tmax | hr*umol/ | hr*umol/ | hr*umol/ | umol/ | Tmax |
| | | PK_ | ject_ | L | L | L | L | hr | L | L | L | L | hr |
| Cohort | Period | DAY | ID | Estimate | | | | | | | | | |
| 1 | 1 | −1 | Mean | 398 | 532 | 673 | 153 | 4.45 | | | | | |
| | | | SD | 82.2 | 148 | 169 | 46.0 | 2.54 | | | | | |
| 1 | 1 | 1 | Mean | 941 | 1120 | 1260 | 395 | 1.15 | 543 | 593 | 584 | 302 | 1.25 |
| | | | SD | 241 | 317 | 376 | 85.0 | 0.580 | 221 | 296 | 359 | 72.1 | 0.589 |
| 1 | 2 | 1 | Mean | 599 | 738 | 833 | 242 | 1.65 | 201 | 206 | 167 | 142 | 1.10 |
| | | | SD | 166 | 198 | 213 | 61.9 | 0.747 | 131 | 169 | 187 | 60.4 | 0.658 |
| 1 | 3 | 1 | Mean | 785 | 954 | 1030 | 296 | 1.60 | 387 | 422 | 359 | 204 | 1.25 |
| | | | SD | 215 | 256 | 271 | 77.7 | 0.810 | 211 | 240 | 251 | 85.5 | 0.677 |
| 1 | 4 | 1 | Mean | 563 | 726 | 823 | 220 | 1.85 | 166 | 194 | 157 | 129 | 1.80 |
| | | | SD | 155 | 191 | 197 | 46.6 | 0.818 | 125 | 157 | 141 | 34.0 | 0.888 |
| 2 | 1 | −1 | Mean | 530 | 749 | 890 | 188 | 3.00 | | | | | |
| | | | SD | 291 | 366 | 401 | 98.0 | 0.943 | | | | | |
| 2 | 1 | 1 | Mean | 1030 | 1260 | 1390 | 398 | 1.35 | 495 | 515 | 499 | 283 | 1.10 |
| | | | SD | 309 | 462 | 525 | 79.0 | 0.747 | 133 | 196 | 223 | 49.2 | 0.615 |

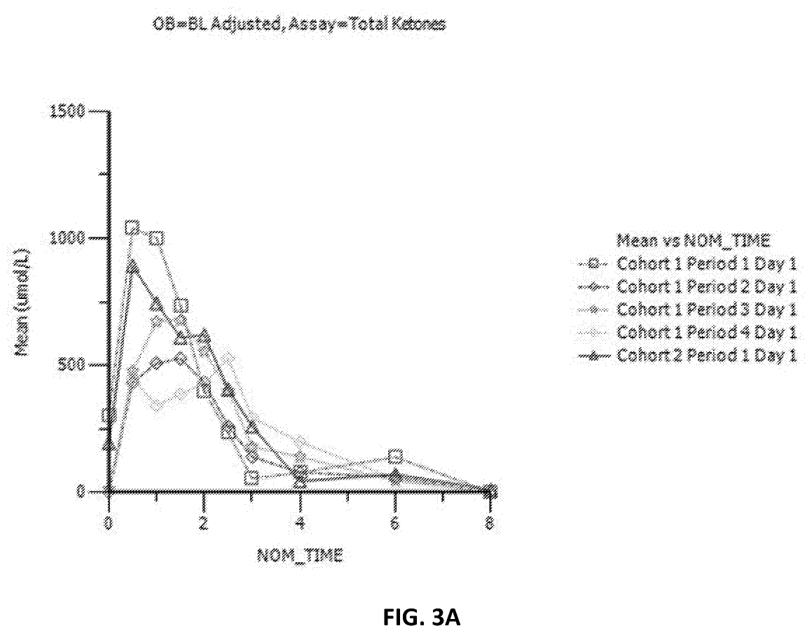

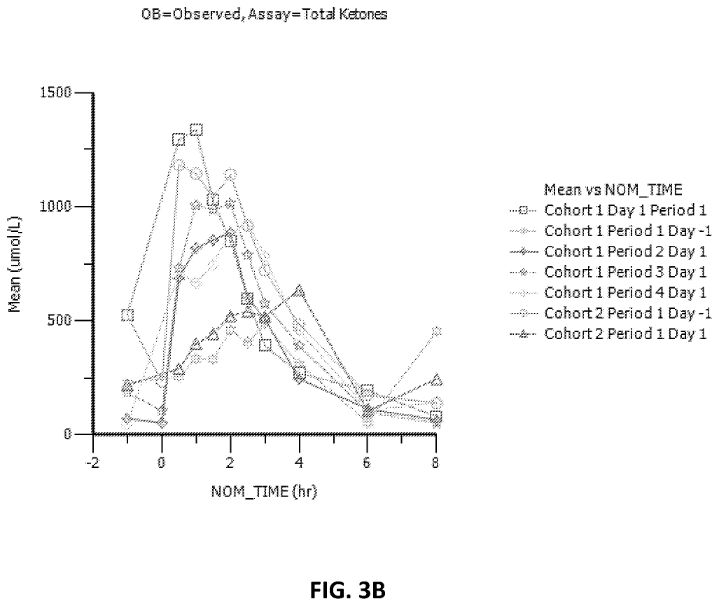

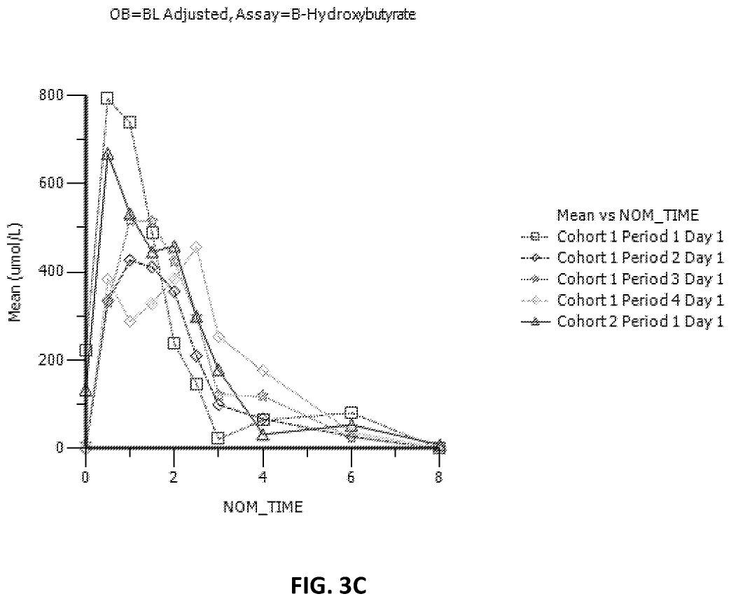

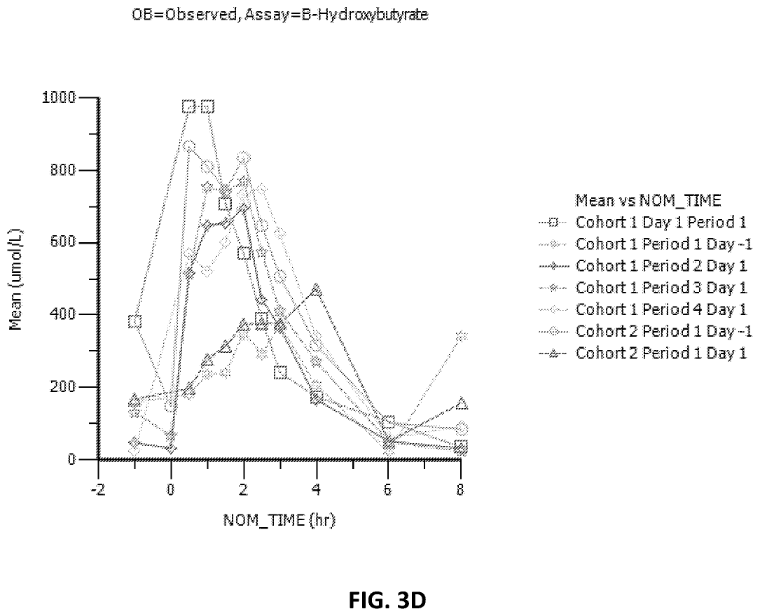

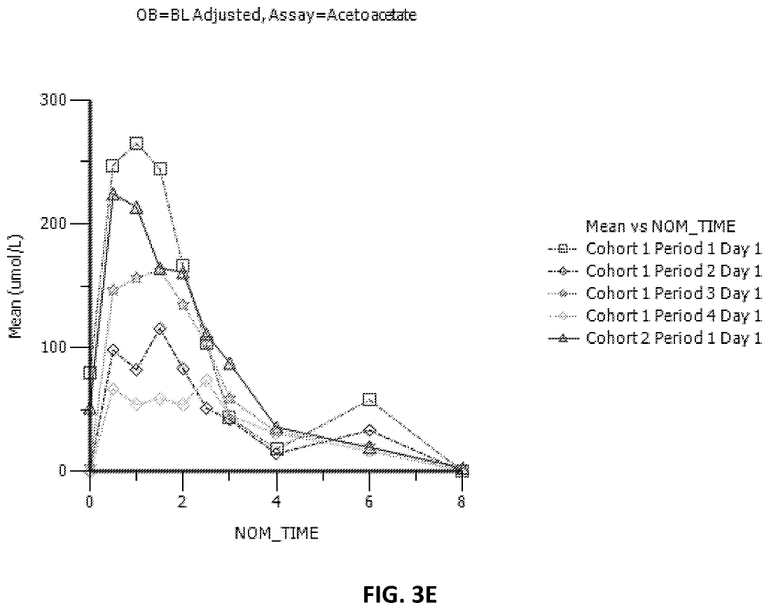

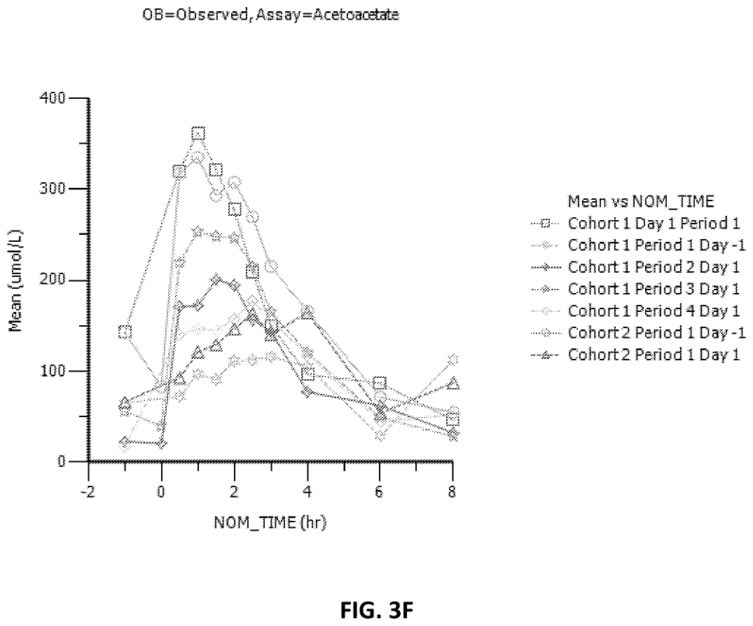

FIGS. 3A-B (total ketone), 3C-D (BHB), and 3E-F (AcAc) show PK values obtained in each cohort after oral administration. The Figures show the best PK was obtained with administration of the MCT with a standard meal. The best PK of those cohorts that were well tolerated showed that administrating the MCT 30 minutes after a high fat meal which was slightly better than administration of the MCT with a standard meal. Fasting while administration of the MCT provided poor tolerability and the lowest PK. In the cohorts with well toleration, the total ketone levels ranged in the 500-1000 μM. Finally, better PK was achieved in the Asian cohort than in the Caucasian cohort. This result is not corrected for BMI (body mass index).

### Example 4—Comparison of Protein Based MCT Formulations Vs. Non-Protein Based MCT Formulations

This study is designed to compare protein based formulations versus non-protein based formulation. Fourteen subjects (healthy young males) were divided into six separate groups. Each group was dosed with one of 6 formulations as shown in Table 5 orally administering 20 g of MCT. The study provided no titration. The study was randomized, open label, and a cross over design.

TABLE 5
Formulation for Study

| | Group | | | | | |
|---|---|---|---|---|---|---|
| | 1 | 2 | 3 | 4 | 5 | 6 |
| | Formulation | | | | | |
| | A | B | C | D | E | F |
| Name | MCT | Milk/ | AC- | AC- | AC- | AC- |
| | Procal | CT | 1207 | 1205 | 1206 | 1202 |
| | | Oil | | | | |
| Com- | | | C8:Non | C8:mod- | C8:Corn | C8:gum |
| position | | | fat milk | ified | Starch:Malto- | acacia |
| | | | (70:28) | starch | dextrin | (33:66) |
| | | | | (50:49) | (64:25:10) | |
| Sup- | Nestle | Stepan | Sensory | Sensory | Sensory | Sensory |
| plier | (Sourced | | Effects | Effects | Effects | Effects |
| | by | | | | | |
| | Bilcare) | | | | | |

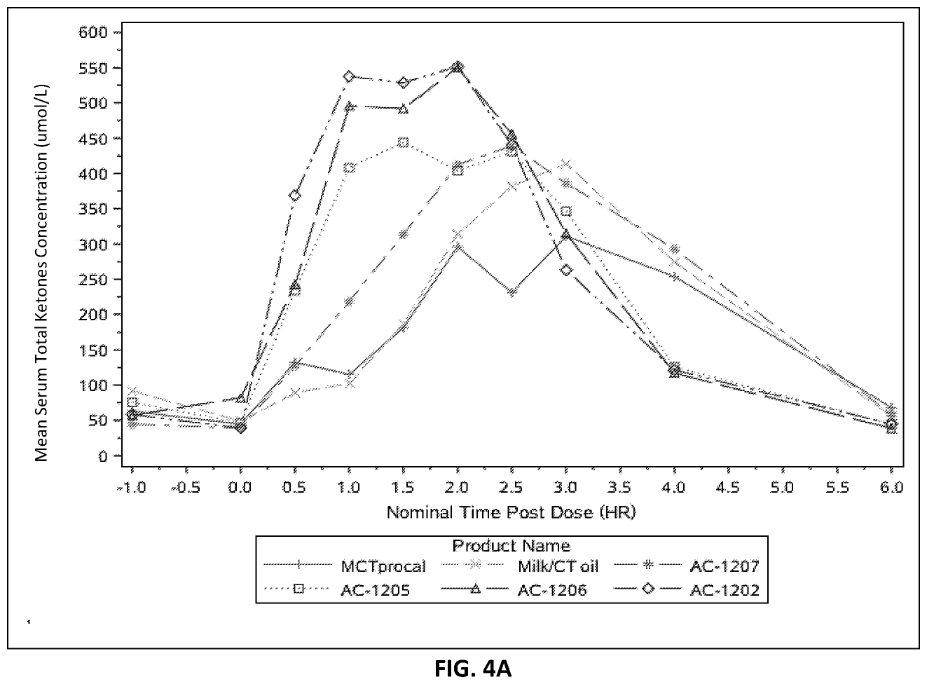

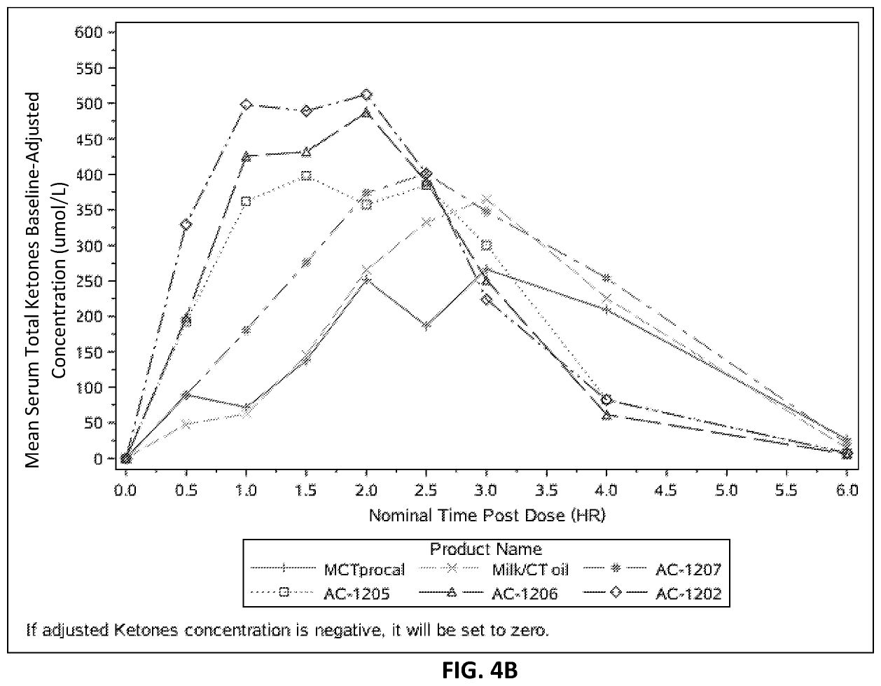

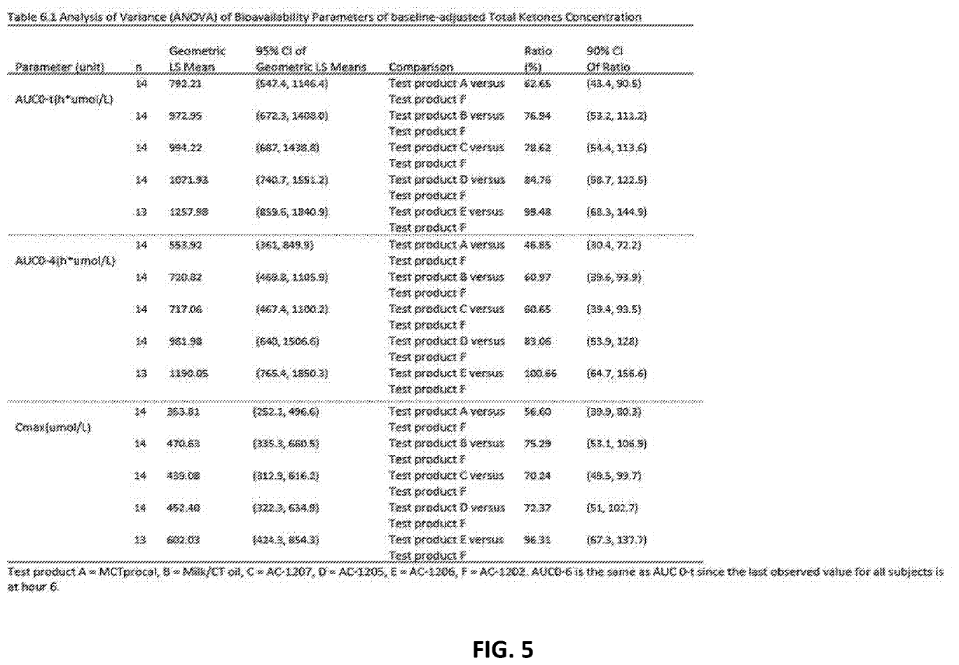

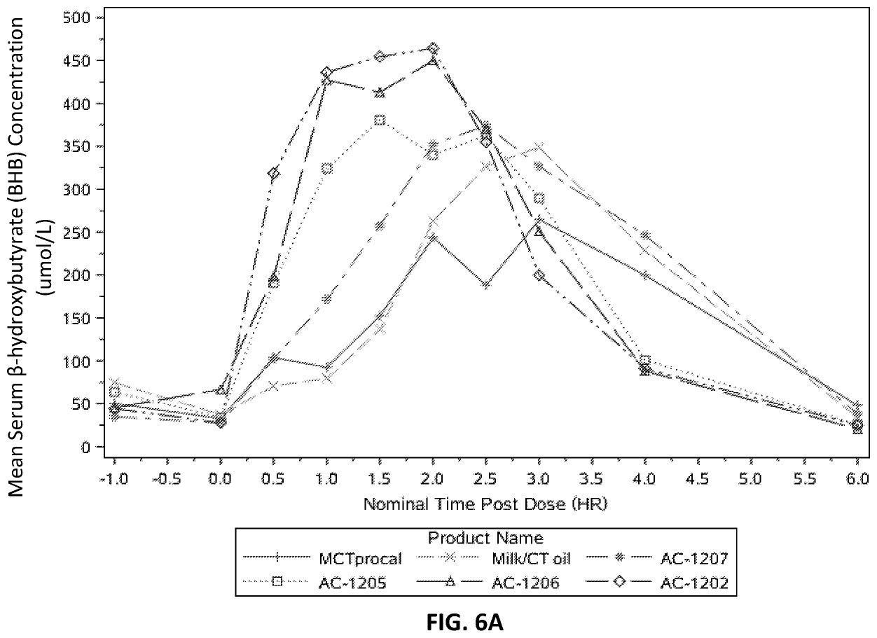

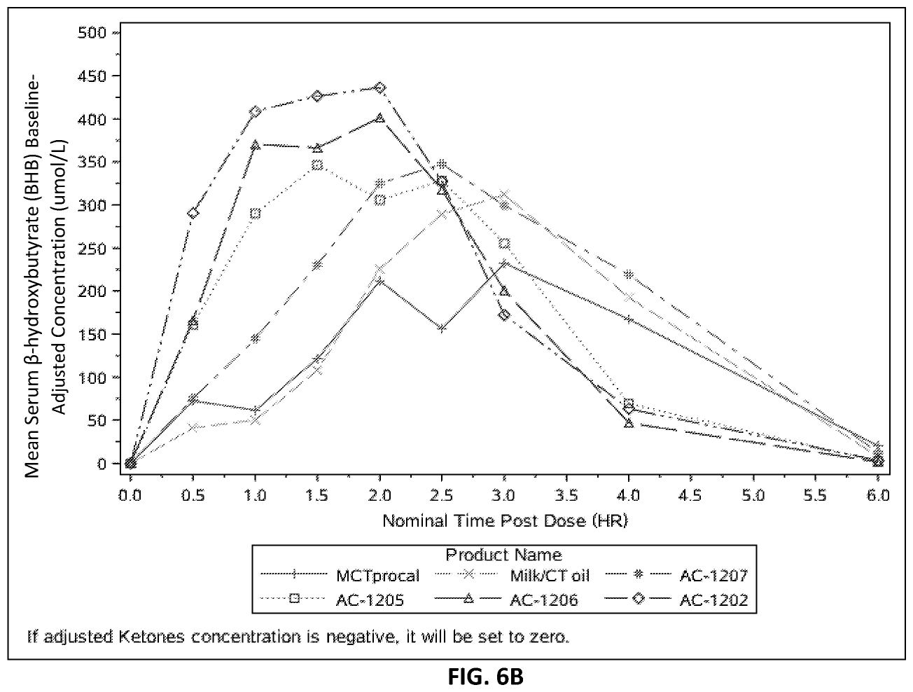

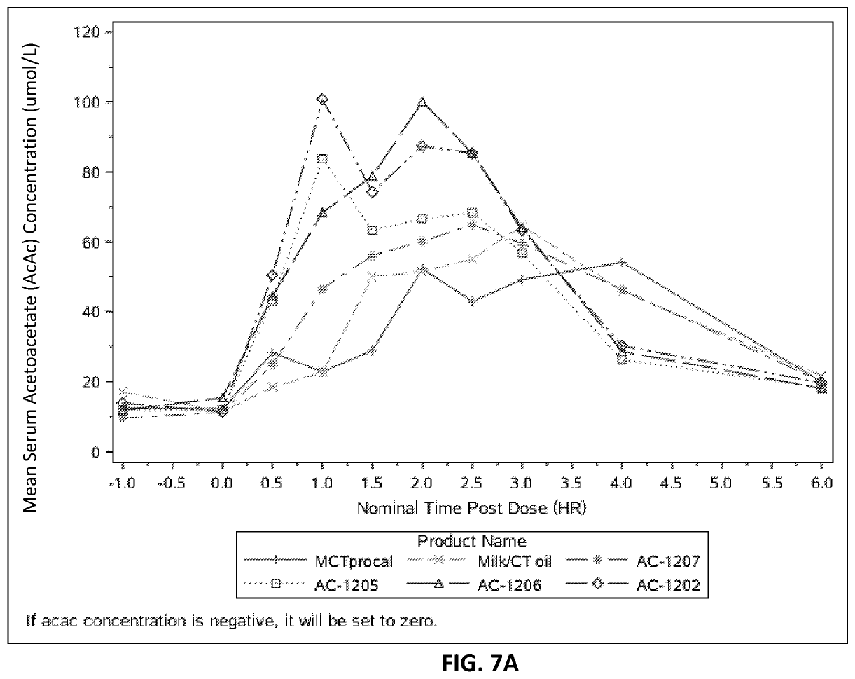

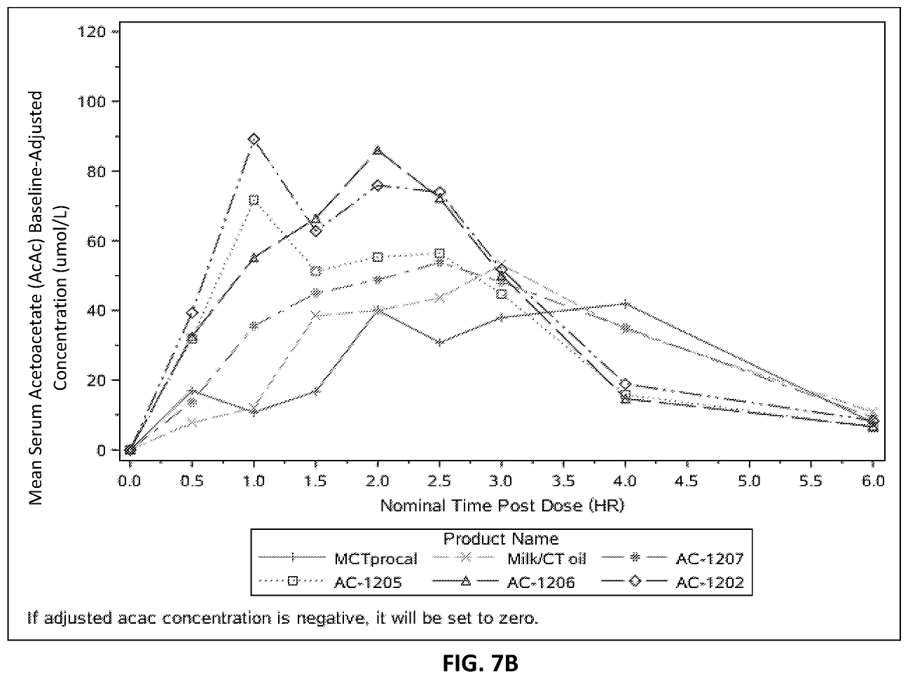

Each formulation was administered orally at hour 0 on day 1 and administered orally approximately 30 minutes after the completion of a standard meal. After administration, blood samples were obtained at various time points from each subject and evaluated using enzymatic methods (Wako Diagnostics) measuring total ketone levels, BHB (β-hydroxybutyrate) levels, and an estimate of acetoacetate (AcAc) levels. The data from these enzymatic assay are shown in FIGS. 4-10. As the above Figures show, AC-1202 achieves the most desirable combination of increased Cₘₐₓ, earliest Tₘₐₓ, and AUC. Protein based formulations (MCT procal, tricapilin/milk-Cunnane and AC-1207) have reasonable AUC, but delayed and lower Cₘₐₓ. The protein formulations appear to delay the release of the MCT. Carbohydrate/gum acacia based formulations provide a more immediate release of the MCT.

1. A method of treating a disease or disorder in a subject in need thereof, comprising administering a therapeutically effective amount medium chain triglycerides (MCT), wherein the therapeutically effective amount of MCT is administered in a pharmaceutical composition, wherein the MCT composition provides a maximum serum concentration (Cmax) of total ketones within at least 3 hours after administration, when administered 30 minutes after a standard meal and in the substantial absence of proteins.

2. The method of claim 1, wherein the MCT composition provides a maximum serum concentration (Cmax) of total ketones within at least 2.5 hours after administration, at least 2 hours after administration, at least 1.5 hours after administration, or at least 1 hour after administration.

3. The method of claim 1, wherein the therapeutically effective amount of MCT is 20 g and wherein the Cmax of total ketones is at least 400 μmol/L, at least 450 μmol/L, or at least 500 μmol/L.

4. A method of treating a disease or disorder in a subject in need thereof, comprising administering a therapeutically effective amount medium chain triglycerides (MCT), wherein the therapeutically effective amount of MCT is administered in a pharmaceutical composition, wherein the MCT composition provides a maximum serum concentration (Cmax) of b-hydroxybutyrate (BHB) within at least 3 hours after administration, when administered 30 minutes after a standard meal and in the substantial absence of proteins.

5. The method of claim 4, wherein the MCT composition provides a maximum serum concentration (Cmax) of BHB within at least 2.5 hours after administration, at least 2 hours after administration, at least 1.5 hours after administration, or at least 1 hour after administration.

6. The method of claim 4, wherein the therapeutically effective amount of MCT is 20 g and wherein the Cmax of BHB is at least 400 μmol/L, at least 450 μmol/L, or at least 500 μmol/L.

7. A method of treating a disease or disorder in a subject in need thereof, comprising administering a therapeutically effective amount medium chain triglycerides (MCT), wherein the therapeutically effective amount of MCT is administered in a pharmaceutical composition, wherein the MCT composition provides a maximum serum concentration (Cmax) of acetoacetate (AcAc) within at least 2.5 hours after administration, when administered 30 minutes after a standard meal and in the substantial absence of proteins.

8. The method of claim 7, wherein the MCT composition provides a maximum serum concentration (Cmax) of AcAc within at least 2 hours after administration, at least 1.5 hours after administration, or at least 1 hour after administration.

9. The method of claim 7, wherein the therapeutically effective amount of MCT is 20 g and wherein the Cmax of AcAc is at least 50 umol/L, at least 60 umol/L, at least 70 umol/L, at least 80 umol/L, at least 90 umol/L, or at least 100 umol/L.

10. The method of claim 1, wherein the MCT pharmaceutical composition is stable at a pH of about 1 to about 3.

11. A method of treating a disease or disorder in a subject in need thereof, comprising administering a therapeutically effective amount medium chain triglycerides (MCT), wherein the therapeutically effective amount of MCT is administered in a pharmaceutical composition, wherein the MCT composition provides a maximum serum concentration (Cmax) of total ketones after at least 2.5 hours after administration, when administered 30 minutes after a standard meal and in the presence of proteins.

12. The method of claim 11, wherein the MCT composition provides a maximum serum concentration (Cmax) of total ketones after at least 3.0 hours after administration, after at least 3.5 hours after administration, after at least 4.0 hours after administration, or after at least 5 hours after administration.

13. The method of claim 11, wherein the therapeutically effective amount of MCT is 20 g and wherein the Cmax of total ketones is at least 200 μmol/L, at least 250 μmol/L, at least 300 μmol/L, or at least 350 μmol/L.

14. A method of treating a disease or disorder in a subject in need thereof, comprising administering a therapeutically effective amount medium chain triglycerides (MCT), wherein the therapeutically effective amount of MCT is administered in a pharmaceutical composition, wherein the MCT composition provides a maximum serum concentration (Cmax) of b-hydroxybutyrate (BHB) after at least 2.5 hours after administration, when administered 30 minutes after a standard meal and in the presence of proteins.

15. The method of claim 14, wherein the MCT composition provides a maximum serum concentration (Cmax) of BHB after at least 3.0 hours after administration, after at least 3.5 hours after administration, after at least 4.0 hours after administration, or after at least 5 hours after administration.

16. The method of claim 14, wherein the therapeutically effective amount of MCT is 20 g and wherein the Cmax of BHB is at least 200 μmol/L, at least 250 μmol/L, at least 300 μmol/L, or at least 350 μmol/L.

17. A method of treating a subject in need thereof, comprising administering a therapeutically effective amount medium chain triglycerides (MCT), wherein the therapeutically effective amount of MCT is administered in a pharmaceutical composition, wherein the MCT composition provides a maximum serum concentration (Cmax) of acetoacetate (AcAc) after at least 2.5 hours after administration, when administered 30 minutes after a standard meal and in the presence of proteins.

18. The method of claim 17, wherein the MCT composition provides a maximum serum concentration (Cmax) of AcAc after at least 3.0 hours after administration, after at least 3.5 hours after administration, after at least 4.0 hours after administration, or after at least 5 hours after administration.

19. The method of claim 17, wherein the therapeutically effective amount of MCT is 20 g and wherein the Cmax of AcAc is at least 20 umol/L, at least 25 umol/L, at least 30 umol/L, at least 35 umol/L, or at least 40 umol/L.

20. The method of claim 11, wherein the MCT pharmaceutical composition is stable at a pH of about 5 to about 7.

21. A method of treating a subject in need thereof, comprising administering a therapeutically effective amount medium chain triglycerides (MCT), wherein the therapeutically effective amount of MCT is administered in two portions,
wherein the first portion comprises a first therapeutically effective amount of medium chain triglycerides (MCTs) that is substantially released within 3 hours upon administration to the subject, and
wherein the second portion comprises a second therapeutically effective amount of medium chain triglycerides (MCTs) and protein, wherein the second amount of MCTs are substantially released from the second portion over 3 or more hours after administration of the second portion to the subject.

22. The method of claim 21, wherein the first portion of MCT is administered in the substantial absence of proteins.

23. The method of claim 21, wherein the first portion of MCT is substantially released within 2.5 hours, within 2 hours, within 1.5 hours, or within 1 hour upon administration to the subject.

24. The method of claim 21, wherein the second portion of MCT is substantially released over 3.5 or more hours, 4 or more hours, 4.5 or more hours, or 5 or more hours upon administration to the subject.

25. The method of claim 1, wherein the disease or disorder is a disease or disorder associated with reduced cognitive function.

26. The method of claim 25, wherein the disease or disorder associated with reduced cognitive function is selected from Alzheimer's Disease and Age-Associated memory impairment.

27. The method of claim 1, wherein the subject lacks the ApoE4 genotype.

28. The method of claim 1, wherein the amount of total ketones, BHB, and/or AcAc is determined using enzymatic methods.

29. A pharmaceutical composition comprising a first component and a second component,
wherein the first component comprises a therapeutically effective amount of a first portion of medium chain triglycerides (MCTs) that is substantially released within 3 hours upon administration of the pharmaceutical composition to a subject in need thereof, and
wherein the second component comprises a therapeutically effective amount of a second portion of medium chain triglycerides (MCTs) and protein, wherein the second portion of MCTs are substantially released from the second component over 3 or more hours after administration of the pharmaceutical composition to the subject.

30. The pharmaceutical composition of claim 29, wherein the first portion of MCTs is substantially released within 2.5 hours, within 2 hours, within 1.5 hours, or within 0.5 hours of administration of the pharmaceutical composition.

31. A pharmaceutical composition, comprising:
medium chain triglycerides (MCTs) and at least one pharmaceutically acceptable excipient, wherein the composition is substantially free of proteins,
wherein the composition provides a maximum serum concentration (Cmax) of at least one ketone body within at least 3 hours after administration, when administered 30 minutes after a standard meal and in the substantial absence of proteins to a subject in need thereof.

32. The composition of claim 31, wherein the MCT composition provides a maximum serum concentration (Cmax) of at least one ketone body within at least 2.5 hours after administration, at least 2 hours after administration, at least 1.5 hours after administration, or at least 1 hour after administration.

33. The composition of claim 31, wherein the at least one ketone body is b-hydroxybutyrate (BHB), acetoacetate (AcAc), or combinations thereof.

34. The composition of claim 31, wherein the MCT pharmaceutical composition is an emulsion.

35. The composition of claim 34, wherein the emulsion does not phase separate for at least 10 minutes, at least 20 minutes, at least 30 minutes, at least 45 minutes, at least 1 hour, at least 2 hours, at least 4 hours, at least 5 hours, or at least 24 hours.

36. The composition of claim 35, wherein the emulsion has a mean droplet diameter of between about 100 nm and about 1000 nm, about 100 nm and about 500 nm, or about 200 nm and about 300 nm.

37. The composition of claim 31, wherein the MCT pharmaceutical composition comprises at least 95% tri:C8 MCT.

38. The composition of claim 31, wherein the MCT pharmaceutical composition comprises at least 98% tri:C8 MCT.

39. The composition of claim 31, wherein the MCT pharmaceutical composition comprises at least 95% caprylic triglyceride.

40. The composition of claim 31, wherein the MCT pharmaceutical composition comprises at least 98% caprylic triglyceride.

41. The composition of claim 31, wherein the subject in thereof is a human.
